(19) United States
(12) Patent Application Publication
(10) Pub. No.: US 2011/0045998 A1
(43) Pub. Date: Feb. 24, 2011

(54) CANDIDATE GENES AND BLOOD BIOMARKERS FOR BIPOLAR MOOD DISORDER, ALCOHOLISM AND STRESS DISORDER

(76) Inventors: Alexander B. Niculescu, Indianapolis, IN (US); Helen Le-Niculescu, Indianapolis, IN (US)

Correspondence Address: BARNES & THORNBURG LLP, 11 SOUTH MERIDIAN, INDIANAPOLIS, IN, 46204, US

(21) Appl. No.: 12/681,154

(22) PCT Filed: Sep. 25, 2008
(86) PCT No.: PCT/US08/77642

§ 371 Date: Nov. 10, 2010

Related U.S. Application Data
(60) Provisional application No. 60/978,185, filed on Oct. 8, 2007.

(51) Int. Cl. C40B 30/04; C12Q 1/68; G01N 33/566; C40B 30/00; C40B 40/08

(52) U.S. Cl. 506/9; 435/6; 435/7.92; 506/7; 506/17; 435/7.21

(57) ABSTRACT

Analysis of the gene expression changes identified a series of novel candidate genes and blood biomarkers for bipolar disorder, alcoholism and stress disorder. These are used for diagnosing the disorders, predicting and monitoring response to treatment. A novel treatment for these co-morbid disorders, DHA (Docosahexaenoic acid—an omega-3 fatty acid) was identified, using these genes and biomarkers, as well as the transgenic animal model.

This application claims priority to 60/978,185, filed Oct. 8, 2007, the disclosure of which is hereby incorporated by reference in its entirety.

Part of the work during the development of this invention was made with government support from the NIH under grant NIMH R01 MH071912-01. The U.S. Government has certain rights in the invention.

### BACKGROUND

Genes and biomarkers for diagnosing various psychiatric disorders are disclosed.

Circadian clock genes are compelling candidates for involvement in bipolar disorders, especially the core clinical phenomenology of cycling and switching from depression to mania. Circadian rhythm and sleep abnormalities have long been described in bipolar disorder—excessive sleep in the depressive phase, reduced need for sleep in the manic phase. Sleep deprivation is one of the more powerful and rapid acting treatment modalities for severe depression, and can lead to precipitation of manic episodes in bipolar patients. Seasonal affective disorder (SAD), a variant of bipolar disorder, is tied to the amount of daylight, which is a primary regulator of circadian rhythms and clock gene expression. Lithium, a treatment option for bipolar disorder, has been implicated in the regulation of the circadian clock.

A clock gene D-box binding protein (Dbp) has been identified as a potential candidate gene for bipolar disorder, using a Bayesian-like approach called Convergent Functional Genomics (CFG), that cross-matches animal model gene expression data with human genetic linkage/association data, as well as human tissue data. The integration of multiple independent lines of evidence, each by itself lacking sufficient discriminatory power, leads to the identification of high probability candidate genes, pathways and mechanisms for the disease of interest. DBP knock-out mice have abnormal circadian and homeostatic aspects of sleep regulation.

To further analyze and validate Dbp as a molecular underpinning of bipolar and related disorders and to identify various genes associated with Dbp signaling as biomarkers, in the present application, behavioral and gene expression studies were conducted in mice with a constitutive homozygous deletion of Dbp (DBP KO mice). Blood gene expression studies were also conducted to identify genes that change concomitantly in brain and blood for identifying candidate biomarkers.

### SUMMARY

Analysis of the gene expression changes identified a series of novel candidate genes and blood biomarkers for bipolar disorder, alcoholism and stress disorder. Identified biomarkers for bipolar disorder and/or co-morbid alcoholism represent novel genes for diagnosing and predicting the psychiatric disorders. Treatment with the omega-3 fatty acid DHA (docosahexaenoic acid) was found to reverse (correct) behavioral and gene expression/blood biomarker abnormalities (Tables 4-6; FIGS. 7 and 8).

A method of diagnosing bipolar disorder, alcoholism and stress disorder in an individual includes: (a) determining the level of a plurality of biomarkers for disorder and/or comorbid alcoholism in a sample from the individual, the plurality of biomarkers selected from the group consisting of biomarkers listed in Table 4 and/or Table 5 and/or 6; and (b) diagnosing the presence or absence of the disorders in the individual based on the level of the plurality of biomarkers, optionally with one or more clinical information, obtained by interviewing the individual.

Some of the biomarkers include a subset of blood biomarkers selected from a group of markers from Tables 4, 5, and 6, a subset of which are designated as Cnp (cyclic nucleotide phosphodiesterase 1), Hnrpdl (heterogeneous nuclear ribonucleoprotein D-like), Ywhaz tyrosine 3-monooxygenase/tryptophan 5-monooxygenase activation protein, zeta polypeptide), Sgk (serum/glucocorticoid regulated kinase), Slc38a2 (solute carrier family 38, member 2), Abhd14a (abhydrolase domain containing 14A), Ap1s2 (adaptor-related protein complex 1, sigma 2 subunit), B230337E12Rik (RIKEN cDNA B230337E12 gene), and Snca (synuclein alpha).

Some of the biomarkers include a subset of biomarkers designated as Drd2 (dopamine receptor 2), Clk1 (CDC-like kinase 1), Itgav (integrin alpha V), Gls (glutaminase), Cnp (cyclic nucleotide phosphodiesterase 1), Hnrpdl (heterogeneous nuclear ribonucleoprotein D-like), Kcnj4 (potassium inwardly-rectifying channel, subfamily J, member 4), Gnb1 (guanine nucleotide binding protein, beta 1), Clic4 (chloride intracellular channel 4), Ywhaz (tyrosine 3-monooxygenase/tryptophan 5-monooxygenase activation protein, zeta polypeptide), Sgk (serum/glucocorticoid regulated kinase), Slc38a2 (solute carrier family 38, member 2), Gpm6b (Glycoprotein M6B), Abhd14a (abhydrolase domain containing 14A), Ap1s2 (adaptor-related protein complex 1, sigma 2 subunit), B230337E12Rik (RIKEN cDNA B230337E12 gene), and Snca (synuclein, alpha).

Some of the biomarkers include a subset of blood biomarkers selected from the group of Cnp (cyclic nucleotide phosphodiesterase 1), Hnrpdl (heterogeneous nuclear ribonucleoprotein D-like), Ywhaz tyrosine 3-monooxygenase/tryptophan 5-monooxygenase activation protein, zeta polypeptide), Sgk (serum/glucocorticoid regulated kinase), Slc38a2 (solute carrier family 38, member 2), Abhd14a (abhydrolase domain containing 14A), Ap1s2 (adaptor-related protein complex 1, sigma 2 subunit), and B230337E12Rik (RIKEN cDNA B230337E12 gene).

Suitable sample may be a bodily fluid and the bodily fluid may be blood. A tissue biopsy sample of the individual is also suitable.

Biomarker presence or levels may be determined by analyzing the expression level of RNA transcripts or by analyzing the level of protein or peptides or fragments thereof using one or more techniques that include for example, microarray gene expression analysis, polymerase chain reaction (PCR), real-time PCR, quantitative PCR, immunohistochemistry, enzyme-linked immunosorbent assays (ELISA), and antibody arrays.

In an embodiment, the determination of the level of the plurality of biomarkers is performed by an analysis of the presence or absence of the biomarkers.

A method of predicting the probable course and outcome (prognosis) of bipolar disorder, alcoholism and/or stress disorder in a subject includes: (a) obtaining a test sample from a subject, wherein the subject is suspected of having bipolar disorder and/or co-morbid alcoholism; (b) analyzing the test sample for the presence or level of a plurality of biomarkers, wherein the markers are selected from the group consisting of biomarkers listed in Tables 4-6; and (c) determining the prognosis of the subject based on the presence or level of the biomarkers and one or more clinicopathological data to implement a particular treatment plan for the subject.

Suitable clinicopathological data include for example, patient age, previous personal and/or familial history of psychiatric illness, psychosis, previous personal and/or familial history of response to medications, and any genetic or biochemical predisposition to psychiatric illness.

Some of the test samples include for example, fresh blood, stored blood, fixed, paraffin-embedded tissue, tissue biopsy, tissue microarray, fine needle aspirates, peritoneal fluid, ductal lavage and pleural fluid or a derivative thereof.

A method of predicting the likelihood of a successful treatment for bipolar disorder, alcoholism and/or stress disorder in a patient includes:

(a) determining the expression level of biomarkers selected from a group of markers listed in Tables 4, or 5 or 6, a subset of which are designated as Cnp (cyclic nucleotide phosphodiesterase 1), Hnrpdl (heterogeneous nuclear ribonucleoprotein D-like), Ywhaz tyrosine 3-monooxygenase/tryptophan 5-monooxygenase activation protein, zeta polypeptide), Sgk (serum/glucocorticoid regulated kinase), Slc38a2 (solute carrier family 38, member 2), Abhd14a (abhydrolase domain containing 14A), Ap1s2 (adaptor-related protein complex 1, sigma 2 subunit), B230337E12Rik (RIKEN cDNA B230337E12 gene), and Snca (synuclein alpha); and

(b) predicting the likelihood of successful treatment for psychosis by determining whether the sample from the patient expresses biomarkers for delusion or hallucination.

A method of treating a patient suspected of suffering bipolar disorder, alcoholism and/or stress disorder includes:
(a) diagnosing whether the patient suffers from bipolar disorder, alcoholism and/or stress disorder by determining the expression level of one or more of the biomarkers listed in Tables 4-6 in a sample obtained from the patient;
(b) selecting a treatment for bipolar disorder, alcoholism and/or stress disorder based on the determination whether the patient suffers from delusion or hallucination; and
(c) administering to the patient a therapeutic agent or a combination of agents capable of treating bipolar disorder, alcoholism and/or stress disorder.

Suitable therapeutic agents include for example, DHA or similar omega-3 fatty acids derivatives. A treatment plan may be personalized plant for the patient depending on the results of biomarker analysis.

A method for clinical screening of agents capable of affecting bipolar disorder, alcoholism and/or stress disorder includes:
(a) administering a candidate agent to a population of individuals suspected of suffering from bipolar disorder, alcoholism and/or stress disorder;
(b) monitoring the expression profile of one or more of the biomarkers listed in Tables 4-6 in blood samples obtained from the individuals receiving the candidate agent compared to a control group; and
(c) determining that the candidate agent is capable of affecting bipolar disorder, alcoholism and/or stress disorder based on the expression profile of one or more of the biomarkers in the blood samples obtained from the individuals receiving the candidate drug compared to the control.

A diagnostic microarray for bipolar disorder, alcoholism and/or stress disorder includes a plurality of nucleic acid molecules representing genes selected from the group of genes listed in Tables 4-6.

A kit for diagnosing bipolar disorder, alcoholism and/or stress disorder includes a component selected from the group consisting of (i) oligonucleotides for amplification of one or more genes listed in Tables 4-6 (ii) immunohistochemical agents capable of identifying the protein products of one or more biomarkers listed in Tables 4-6 (iii) a microarray having a plurality of markers listed in Tables 4-6, and (iv) a biomarker expression index representing the genes listed in Tables 4-6 for correlation.

A diagnostic microarray includes a panel of biomarkers that are predictive of bipolar disorder, alcoholism and/or stress disorder, wherein the microarray includes nucleic acid fragments representing biomarkers listed Tables 4, 5, and 6, a subset of which are designated as Cnp (cyclic nucleotide phosphodiesterase 1), Hnrpdl (heterogeneous nuclear ribonucleoprotein D-like), Ywhaz tyrosine 3-monooxygenase/tryptophan 5-monooxygenase activation protein, zeta polypeptide), Sgk (serum/glucocorticoid regulated kinase), Slc38a2 (solute carrier family 38, member 2), Abhd14a (abhydrolase domain containing 14A), Ap1s2 (adaptor-related protein complex 1, sigma 2 subunit), B230337E12Rik (RIKEN cDNA B230337E12 gene), and Snca (synuclein alpha).

A transgenic DBP-knockout mouse, wherein the genetic background of the mouse is C57/BL6.

A method for treating bipolar disorder, alcoholism and stress disorder, wherein the treatment is DHA or other omega-3 fatty acids, administered orally or in injectable form.

### BRIEF DESCRIPTION OF THE DRAWINGS

FIG. 1 demonstrates phenomics—locomotion at baseline in DBP KO NST mice. (a) Total Distance Traveled; (b) Mean Spatial Deviance. Error bars on histograms represent standard-error of mean (SEM). Graphs of data for males and both genders combined are shown. Genotypes x Drug p-values are derived from two-way analyses of variance (ANOVAs), as described herein. * represents individual comparison p-values derived from t-test. For males, p=0.0246; for combined group, p=0.01.

FIG. 2 shows phenomics of DBP KO ST mice: locomotion, switch, sleep deprivation, clustering. (a) After the 28 day stress (ST) paradigm video tracking software was used to measure the mean total distance traveled during a 30 minute period in both wild type and knockout mice with and without methamphetamine treatment (* p-value=0.0369); (b) Stress-induced switch in total distance traveled comparing wild type and knockout mice (* p-value=0.01583); (c) Sleep deprivation caused an increase in the total distance traveled by DBP KO mice compared to wild type controls. This increase in locomotion is prevented by pretreatment with valproate (* p-value=0.0068); (d) group, and (e) individual mice. Clustering of video-tracker data using a PhenoChipping approach31, as described herein.

FIG. 3 demonstrates phenomics—weight (a) Wild-type and DBP KO NST (non-stressed) mice (group housed) (b) Wild-type and DBP KO ST (Stressed) mice (single housed). Body weight measurements were taken at various time points. Data (n) is representative of a mixed population of repeated and individual measures of weight from animals at different time points. Scatter plots of data collected are shown. The best fit line for each set of data was determined and is displayed along with the equation for the line and the R2 value.

FIG. 4 shows phenomics—(a) ethanol and (b) sucrose consumption during the ST paradigm. (a) Alcohol free-choice drinking paradigm, male and female, wild type and DBP KO mice. Fluid consumption from both bottles was monitored for a period of 30 days with an acute stressor at the end of the third week, as described herein; (b) Average sucrose consumption in a separate cohort undergoing the same stress paradigm. Two way ANOVAs were preformed on all data sets. * represents significant p<0.05 by ANOVA.

FIG. 5 illustrates Expanded Convergent Functional Genomics (CFG) analysis. Bayesian integration of multiple animal model and human lines of evidence.

FIG. 6 represents a subset of candidate genes. (a) DBP KO NST; (b) DBP KO ST based on Tables 4 and 5. Scoring of lines of evidence depicted on the side of the pyramid.

FIG. 7 shows behavioral testing of DBP KO and WT mice undergoing a stress (ST) paradigm (a-d). Effects of High DHA vs. Low DHA diet. DHA has a normalizing effect (reverses) some of the behavioral abnormalities (differences) seen between DBP KO ST mice and WT mice.

FIG. 8 shows effects of High DHA vs. Low DHA diet on alcohol consumption in DBP KO ST mice. DHA reverses the behavioral abnormalities (differences) seen between DBP KO ST mice and WT mice. DHA has a normalizing effect (decreases) the increased alcohol consumption seen in these mice.

### DETAILED DESCRIPTION

Phenomic studies and a comprehensive Convergent Functional Genomics approach were used in a knock-out mouse to identify biomarkers for bipolar disorder and alcoholism. These studies revealed that knocking out DBP leads to a phenotype that is germane to bipolar disorder and alcoholism. Moreover, the phenotype is modulated by behavioral stress. Stress is a major precipitant of bipolar disorder episodes in human patients, and increased alcohol consumption in alcoholics. Microarray studies in the prefrontal cortex (PFC) and amygdala (AMY) of mice lacking DBP vs. wild-type littermate control mice, with or without exposure to stress, revealed the underlying cascades of gene expression changes to identify candidate genes, pathways and mechanisms for bipolar, alcoholism, and related disorders. Furthermore, blood gene expression studies in animals identified genes that change concomitantly in brain and blood, and thus represent biomarkers for diagnostic and prognostic clinical uses.

The DBP KO mice are a constitutive knock-out and provides a suitable equivalent of the human bipolar disorder genetic scenario, where most mutations are likely constitutive rather than acquired, as reflected in the familial inheritance of the disorder. The mice colony used herein is on a mixed genetic background, generated by heterozygote breeding provides a suitable model of the human condition, which occurs at a population level in a mixed genetic background.

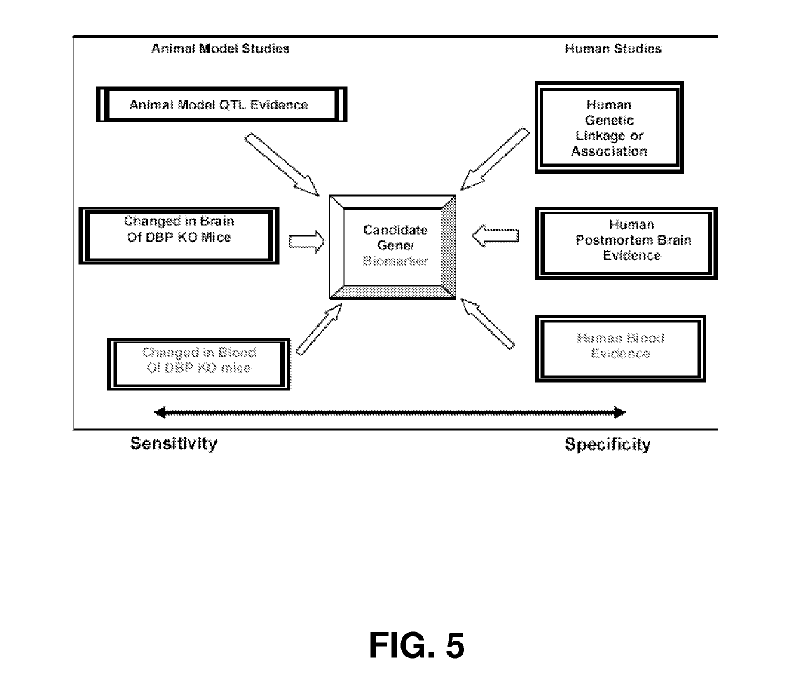

Data presented herein provide an ascertainment of certain phenotypic features, and the gene expression changes underpinning them. Microarray gene expression data from individual mice were compared from experiments performed at 3 different times, with different batches of mice (three mice per genotype per condition). Only the genes that were reproducibly changed in the same direction in at least 6 out of 9 independent comparisons were considered. This overall design was geared to factor out both biological and technical variability. Analytical techniques adopted herein are based on the concordance of multiple tissues (PFC, AMY, blood), each of which are independent microarray experiments, and has multiple additional external Bayesian cross-validators, including human data, for each gene that is called reproducibly changed in the KO mice. Top candidate genes, for which there are multiple independent lines of evidence, are less likely to be false positives. The network of lines of evidence for each gene is resilient, even if one or another of the nodes (lines of evidence) is less than optimal. Convergent Functional Genomics approach was used to extract signal and prioritize findings from large and potentially noisy datasets (FIG. 5). For example, that Snca, a gene associated with alcohol craving in humans is identified as a gene and blood biomarker in the activated, increased alcohol consuming DBP KO ST mice.

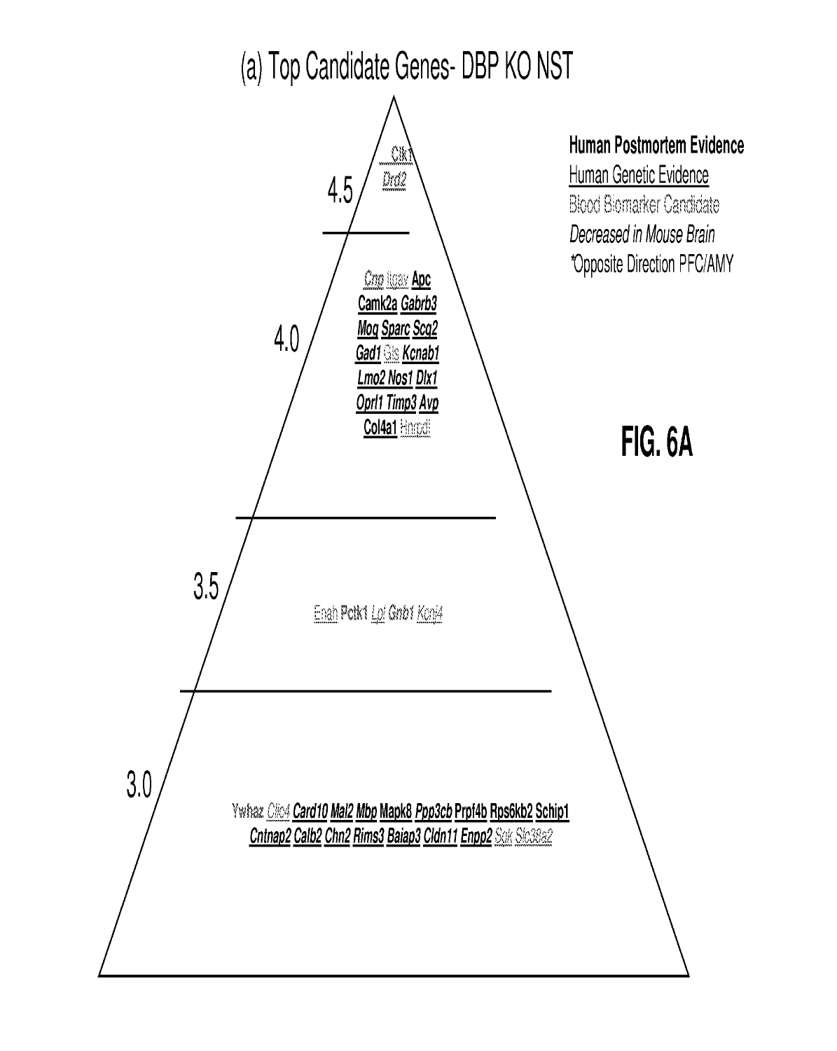

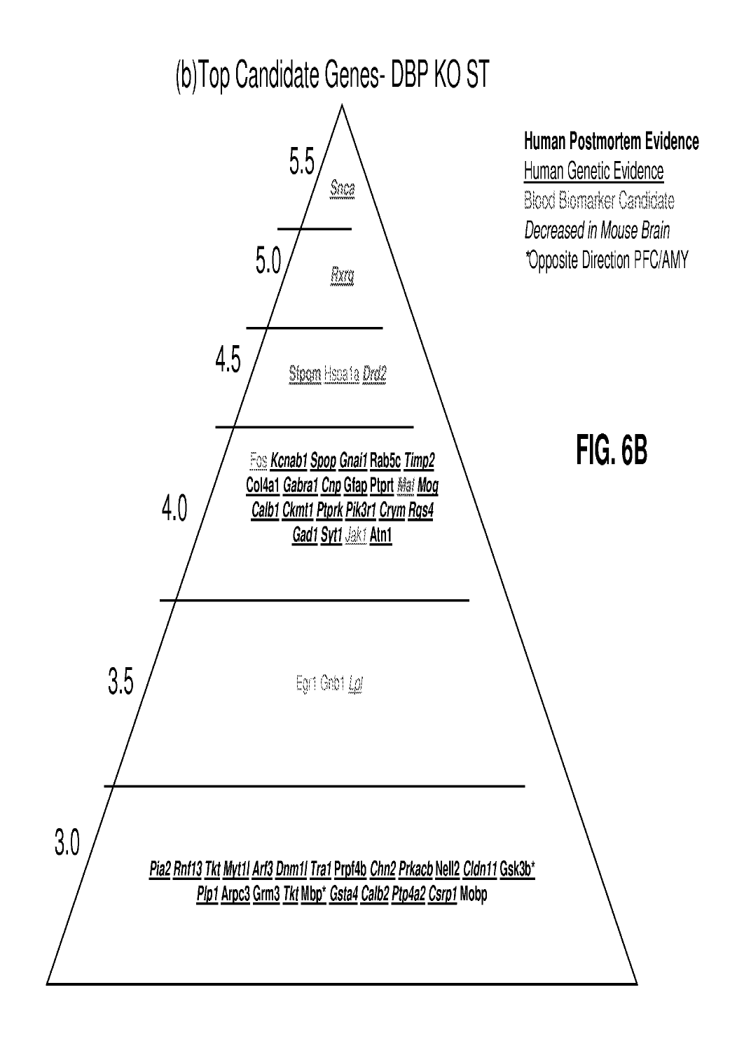

The results presented herein provide direct implications for clinical uses. The behavioral phenomenology and inferences from molecular changes in the DBP knock-out mice bear striking resemblances to DSM criteria for bipolar disorder. Response to stress and switch in phenotype is a cardinal aspect of the human condition. Therefore, the knock-out mice described herein is a first genetic animal models of bipolar disorder to be described. New candidate genes as biomarkers, pathways and mechanisms for bipolar and related disorders were uncovered, including additional clock genes. These prioritized candidates (FIG. 6, Tables 4-6) are useful for extracting signal from whole-genome association studies. Some of the genes identified may be directly regulated by DBP through promoter binding, while others may be regulated indirectly by a cascade of gene expression changes set in motion by DBP. Data presented herein provide support for an underlying non-specific glia/myelin hypofunction and inflammatory/neurodegenerative phenomenology in bipolar and related disorders, both of which may contribute to a functional hypofrontality leading to affective and hedonic dysregulation. The data and analysis presented herein are the first comprehensively analytical approach at brain-blood correlations in an animal model, and integrate that with other multiple lines of evidence, as a way of identifying and prioritizing candidate blood biomarkers for psychiatric disorders. Some of the candidate genes in the dataset encode for proteins that are modulated by existing pharmacological agents (Table 7), which may provide a basis for avenues for rational polypharmacy using currently available agents. For drug development, DBP KO mice may serve a useful role for pre-clinical studies and validation of new candidate drugs for bipolar and related disorders. The insights into overlapping phenomics, genomics and biomarkers among bipolar, alcoholism, stress and related disorders provided by this mouse model point in a translational fashion to the issue of heterogeneity, overlap and interdependence of major psychiatric syndromes as currently defined by DSM, and the need for a move towards comprehensive empirical profiling and away from categorical diagnostic classifications.

A panel of 10 or 20 biomarkers, as illustrated in an example described herein, is a suitable subset that is useful in diagnosing a mood disorder. Larger subsets that includes for example, 150, 200, 250, 300, 350, 400, 450, or 500 markers are also suitable. Smaller subsets that include high-value markers including about 2, 5, 10, 15, 20, 25, 50, 75, and 100 are also suitable. A variable quantitative scoring scheme can be designed using any standard algorithm, such as a variable selection or a subset feature selection algorithms can be used. Both statistical and machine learning algorithms are suitable in devising a frame work to identify, rank, and analyze association between marker data and phenotypic data (e.g., mood disorders).

The term “predictive” or the term “prognostic” does not imply 100% predictive ability. The use of these terms indicates that subjects with certain characteristics are more likely to experience a clinically positive outcome than subjects who do not have such characteristics. For example, characteristics that determine the outcome include one or more of the biomarkers for the psychiatric disorder disclosed herein. Certain conditions are identified herein as associated with an increased likelihood of a clinically positive outcome, e.g., biomarkers for psychiatric disorder and the absence of such conditions or markers will be associated with a reduced likelihood of a clinically positive outcome.

The phrase “clinically positive outcome” refers to biological or biochemical or physical or physiological responses to treatments or therapeutic agents that are generally prescribed for that condition compared to a condition would occur in the absence of any treatment. A “clinically positive outcome” does not necessarily indicate a cure, but could indicate a lessening of symptoms experienced by a subject.

The term “marker” or “biomarker” as used herein, refers to nucleic acid sequences or proteins or polypeptides or fragments thereof to be used for associating a disease state with the marker. Nucleic acids or proteins or polypeptides or portions thereof used as markers are contemplated to include any fragments thereof, in particular, fragments that can specifically hybridize with their intended targets under stringent conditions and immunologically detectable fragments. One or more markers may be related. Marker may also refer to a gene or DNA sequence having a known location on a chromosome and associated with a particular gene or trait. Genetic markers associated with certain diseases or for pre-disposing disease states can be detected in the blood and used to determine whether an individual is at risk for developing a disease. Levels of gene expression and protein levels are quantifiable and the variation in quantification or the mere presence or absence of the expression may also serve as markers.

As used herein, “array” or “microarray” refers to an array of distinct polynulceotides, oligonucleotides, polypeptides, or oligopeptides synthesized on a substrate, such as paper, nylon, or other type of membrane, filter, chip, glass slide, or any other suitable solid support. Arrays also include a plurality of antibodies immobilized on a support for detecting specific protein products. There are several microarrays that are commercially available. A microarray may include one or more biomarkers disclosed herein. A panel of about 20 biomarkers as nucleic acid fragments can be included in an array. The nucleic acid fragments may include oligonucleotides or amplified partial or complete nucleotide sequences of the biomarkers.

In an embodiment, the microarray is prepared and used according to the methods described in U.S. Pat. No. 5,837,832, Chee et al.; PCT application WO95/11995, Chee et al.; Lockhart et al., 1996. Nat Biotech., 14:1675-80; and Schena et al., 1996. Proc. Natl. Acad. Sci. 93:10614-619, all of which are herein incorporated by reference to the extent they relate to methods of making a microarray. Arrays can also be produced by the methods described in Brown et al., U.S. Pat. No. 5,807,522. Arrays and microarrays may be referred to as “DNA chips” or “protein chips.”

“Therapeutic agent” means any agent or compound useful in the treatment, prevention or inhibition of mood disorder or a mood-related disorder.

The term “condition” refers to any disease, disorder or any biological or physiological effect that produces unwanted biological effects in a subject.

The term “subject” refers to an animal, or to one or more cells derived from an animal. The animal may be a mammal including humans. Cells may be in any form, including but not limited to cells retained in tissue, cell clusters, immortalized cells, transfected or transformed cells, and cells derived from an animal that have been physically or phenotypically altered.

Any body fluid of an animal can be used in the methods of the invention. Suitable body fluids include a blood sample (e.g., whole blood, serum or plasma), urine, saliva, cerebrospinal fluid, tears, semen, and vaginal secretions. Also, lavages, tissue homogenates and cell lysates can be utilized.

Many different methods can be used to determine the levels of markers. For example, protein arrays, protein chips, cDNA microarrays or RNA microarrays are suitable. More specifically, one of ordinary skill in the art will appreciate that in one example, a microarray may comprise the nucleic acid sequences representing genes listed in Table 1. For example, functionality, expression and activity levels may be determined by immunohistochemistry, a staining method based on immunoenzymatic reactions uses monoclonal or polyclonal antibodies to detect cells or specific proteins. Typically, immunohistochemistry protocols include detection systems that make the presence of markers visible (to either the human eye or an automated scanning system), for qualitative or quantitative analyses. Mass-spectrometry, chromatography, real-time PCR, quantitative PCR, probe hybridization, or any other analytical method to determine expression levels or protein levels of the markers are suitable. Such analysis can be quantitative and may also be performed in a high-through put fashion. Cellular imaging systems are commercially available that combine conventional light microscopes with digital image processing systems to perform quantitative analysis on cells and tissues, including immunostained samples. (See e.g. the CAS-200 System (Becton, Dickinson & Co.)). Some other examples of methods that can be used to determine the levels of markers include immunohistochemistry, automated systems, quantitative IHC, semi-quantitative IHC and manual methods. Other analytical systems include western blotting, immunoprecipitation, fluorescence in situ hybridization (FISH), and enzyme immunoassays.

The term “diagnosis”, as used in this specification refers to evaluating the type of disease or condition from a set of marker values and/or patient symptoms where the subject is suspected of having a disorder. This is in contrast to disease predisposition, which relates to predicting the occurrence of disease before it occurs, and the term “prognosis”, which is predicting disease progression in the future based on the marker levels/patterns.

The term “correlating,” as used in this specification refers to a process by which one or more biomarkers are associated to a particular psychiatric disorder. This relationship or association can be determined by comparing biomarker levels in a subject to levels obtained from a control population, e.g., positive control—diseased (with symptoms) population and negative control—disease-free (symptom-free) population. The biomarkers disclosed herein provide a statistically significant correlation to diagnosis at varying levels of probability. Subsets of markers, for example a panel of about 20 markers, each at a certain level range which might be a simple threshold, are said to be correlative or associative with one of the disease states. Such a panel of correlated markers can be then be used for disease detection, diagnosis, prognosis and/or treatment outcome. Preferred methods of correlating markers is by performing marker selection by any appropriate scoring method or by using a standard feature selection algorithm and classification by known mapping functions. A suitable probability level is a 5% chance, a 10% chance, a 20% chance, a 25% chance, a 30% chance, a 40% chance, a 50% chance, a 60% chance, a 70% chance, a 75% chance, a 80% chance, a 90% chance, a 95% chance, and a 100% chance. Each of these values of probability is plus or minus 2% or less. A suitable threshold level for markers of the present invention is about 25 pg/mL, about 50 pg/mL, about 75 pg/mL, about 100 pg/mL, about 150 pg/mL, about 200 pg/mL, about 400 pg/mL, about 500 pg/mL, about 750 pg/mL, about 1000 pg/mL, and about 2500 pg/mL.

Prognosis methods disclosed herein that improve the outcome of a disease by reducing the increased disposition for an adverse outcome associated with the diagnosis. Such methods may also be used to screen pharmacological compounds for agents capable of improving the patient's prognosis, e.g., test agents for psychiatric disorders disclosed herein.

The analysis of a plurality of markers, for example, a panel of about 20 or 10 markers may be carried out separately or simultaneously with one test sample. Several markers may be combined into one test for efficient processing of a multiple of samples. In addition, one skilled in the art would recognize the value of testing multiple samples (for example, at successive time points) from the same individual. Such testing of serial samples may allow the identification of changes in marker levels over time or within a period of interest.

In an embodiment, a prediction score for each subject is derived based on the presence or absence of e.g., 10 biomarkers of the panel in their blood. Each of the 10 biomarkers gets a score of 1 if it is detected as “present” (P) in the blood form that subject, 0.5 if it is detected as “marginally present” (M), and 0 if it is called “absent” (A). The ratio of the sum of the high mood biomarker scores divided by the sum of the low mood biomarker scores is multiplied by 100, and provides a prediction score. If the ratio of high biomarker genes to low mood biomarker genes is 1, i.e. the two sets of genes are equally represented, the mood prediction score is 1×100=100. The higher this score, the higher the predicted likelihood that the subject will have high mood. The predictive score was compared with actual self-reported mood scores in a primary cohort of subjects with a diagnosis of bipolar mood disorder. A prediction score of 100 and above had a 84.6% sensitivity and a 68.8% specificity for predicting high mood. A prediction score below 100 had a 76.9% sensitivity and 81.3% specificity for predicting low mood. The term “present” indicates that a particular biomarker is expressed to a detectable level, as determined by the technique used. For example, in an experiment involving a microarray or gene chip obtained from a commercial vendor Affymetrix (Santa Clara, Calif.), the embedded software rendered a “present” call for that biomarker. The term “present” refers to a detectable presence of the transcript or its translated protein/peptide and not necessarily reflects a relative comparion to for example, a sample from a normal subject. In other words, the mere presence or absence of a biomarker is assigned a value, e.g., 1 and a prediction score is calculated as described herein. The term “marginally present: refers to border line expression level that may be less intense than the “present” but statistically different from being marked as “absent” (above background noise), as determined by the methodology used.

In an embodiment, a prediction score based on differential expression (instead of “present”, “absent”) is used. For example, if a subject has a plurality of markers for high or low mood are differentially expressed, a prediction based on the differential expression of markers is determined. Differential expression of about 1.2 fold or 1.3 or 1.5 or 2 or 3 or 4 or 5-fold or higher for either increased or decreased levels can be used. Any standard statistical tool such as ANOVA is suitable for analysis of differential expression and association with high or low mood diagnosis or prediction.

A prediction based on the analysis of either high or low mood markers alone (instead of a ratio of high versus low mood markers) may also be practiced. If a plurality of high mood markers (e.g., about 6 out of 10 tested) are differentially expressed to a higher level compared to the low mood markers (e.g., 4 out of 10 tested), then a prediction or diagnosis of high mood status can be made by analyzing the expression levels of the high mood markers alone without factoring the expression levels of the low mood markers as a ratio.

In an embodiment, a detection algorithm uses probe pair intensities to generate a detection p-value and assign a Present, Marginal, or Absent call. Each probe pair in a probe set is considered as having a potential vote in determining whether the measured transcript is detected (Present) or not detected (Absent). The vote is described by a value called the Discrimination score [R]. The score is calculated for each probe pair and is compared to a predefined threshold Tau. Probe pairs with scores higher than Tau vote for the presence of the transcript. Probe pairs with scores lower than Tau vote for the absence of the transcript. The voting result is summarized as a p-value. The greater the number of discrimination scores calculated for a given probe set that are above Tau, the smaller the p-value and the more likely the given transcript is truly Present in the sample. The p-value associated with this test reflects the confidence of the Detection call.

Regarding detection p-value, a two-step procedure determines the Detection p-value for a given probe set. The Discrimination score [R] is calculated for each probe pair and the discrimination scores are tested against the user-definable threshold Tau. The detection Algorithm assesses probe pair saturation, calculates a Detection p-value, and assigns a Present, Marginal, or Absent call. In an embodiment, the default thresholds of the Affymetrix MAS 5 software were used.

In spiking experiments by the manufacturer to establish default thresholds (adding of known quantities of test transcripts to a mixture, to measure the sensitivity of the Affymetrix MAS 5 detection algorithm) 80% of spiked transcripts are called Present at a concentration of 1.5 pM. This concentration corresponds to approximately one transcript in 100,000 or 3.5 copies per cell. The false positive rate of making a Present call was roughly 10%, as noted by 90% of the transcripts being called Absent when not spiked into the sample (0 pM concentration).

In another embodiment, a kit for the analysis of markers includes for example, devises and reagents for the analysis of at least one test sample and instructions for performing the assay. Optionally, the kits may contain one or more means for using information obtained from marker assays performed for a marker panel to diagnose mood disorders. Probes for markers, marker antibodies or antigens may be incorporated into diagnostic assay kits depending upon which markers are being measured. A plurality of probes may be placed in to separate containers, or alternatively, a chip may contain immobilized probes. In an embodiment, another container may include a composition that includes an antigen or antibody preparation. Both antibody and antigen preparations may preferably be provided in a suitable titrated form, with antigen concentrations and/or antibody titers given for easy reference in quantitative applications.

The kits may also include a detection reagent or label for the detection of specific reaction between the probes provided in the array or the antibody in the preparation for immunodetection. Suitable detection reagents are well known in the art as exemplified by fluorescent, radioactive, enzymatic or otherwise chromogenic ligands, which are typically employed in association with the nucleic acid, antigen and/or antibody, or in association with a secondary antibody having specificity for first antibody. Thus, the reaction is detected or quantified by means of detecting or quantifying the label. Immunodetection reagents and processes suitable for application in connection with the novel methods of the present invention are generally well known in the art.

The reagents may also include ancillary agents such as buffering agents and protein stabilizing agents, e.g., polysaccharides and the like. The diagnostic kit may further include where necessary agents for reducing background interference in a test, agents for increasing signal, software and algorithms for combining and interpolating marker values to produce a prediction of clinical outcome of interest, apparatus for conducting a test, calibration curves and charts, standardization curves and charts, and the like.

The pharmaceutical compositions and dosage forms of DHA or an omega 3 fatty acid equivalent thereof described herein may optionally comprise one or more additives. Preferred additives include surfactants and polymers. In addition, the composition is not limited with regard to its form, but it is preferred that the formulation is in solid or semi-solid form. Furthermore, the DHA in the pharmaceutical composition may be completely solubilized or partially solubilized and partially suspended in the composition.

In another embodiment, a dosage form of DHA is provided comprising the aforementioned pharmaceutical composition. The dosage form contains a therapeutically effective amount of DHA, preferably in an amount of about 100 to about 2500 mg, and more preferably in an amount of about 100 to about 500 mg. Other suitable doses of DHA include for example, 200, 300, 400, 500, 600, 700, 800, 900, 1000, 1500, 2000, 2500, 3000 and 5000 milligrams. Although the dosage form may be any suitable dosage form, the dosage form of DHA is preferably a capsule containing the pharmaceutical composition having a therapeutically effective amount of DHA contained therein.

A skilled artisan can readily obtain the nucleic acid sequence information for the various genes listed in Tables 4-6 using the Entrez ID provided therein. Accordingly, probes or primers are readily generated for analysis. Microarrays having oligonucleotide probes or other fragments representing one or more of the genes listed in Tables 4-6 are obtained using a commercial source or generated in a laboratory.

### EXAMPLES

The following examples are to be considered as exemplary and not restrictive or limiting in character and that all changes and modifications that come within the spirit of the disclosure are desired to be protected.

### Example 1

### Phenomic Studies: Behavioral Phenotype, Response to Stress and Sleep Deprivation

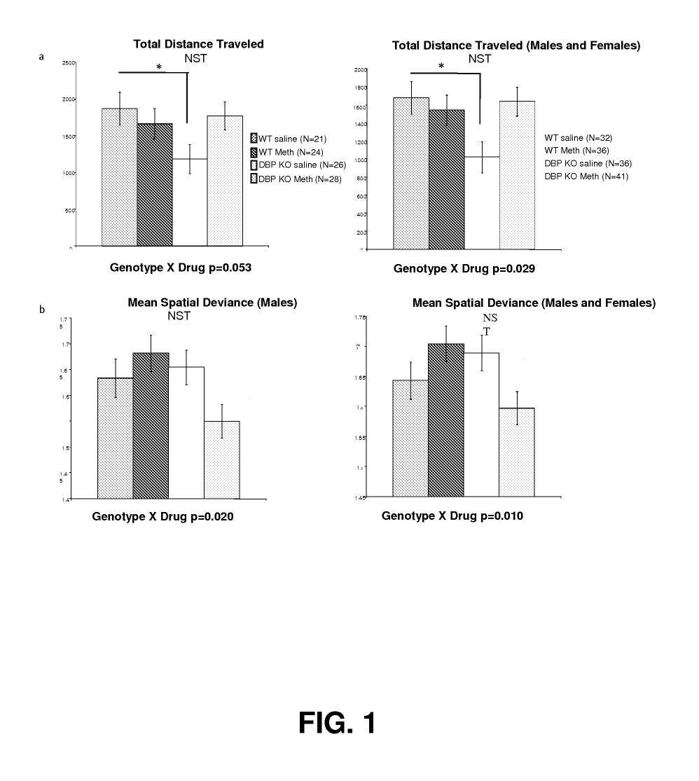

At baseline, DBP KO NST (non-stressed) mice exhibited an overall decrease in the distance traveled as compared to wild type animals. Treatment with methamphetamine reversed this decrease (FIG. 1a). This observation of decreased locomotion in KO mice, along with reported sleep EEG abnormalities indicates that the DBP KO mice at baseline have phenotypic similarities to the depressive phase of bipolar disorder. The KO mice treated with methamphetamine displayed a reduction in stereotypy, as measured by Spatial Deviance, whereas the WT mice exhibited a trend towards an increase in stereotypy (FIG. 1b), which likely accounts for their apparent lack of increased distance traveled. Stereotypy is associated with a strong response to methamphetamine. Thus, at similar doses, KO mice displayed a lower (blunted) response to methamphetamine compared to WT mice, consistent with a lower hedonic state.

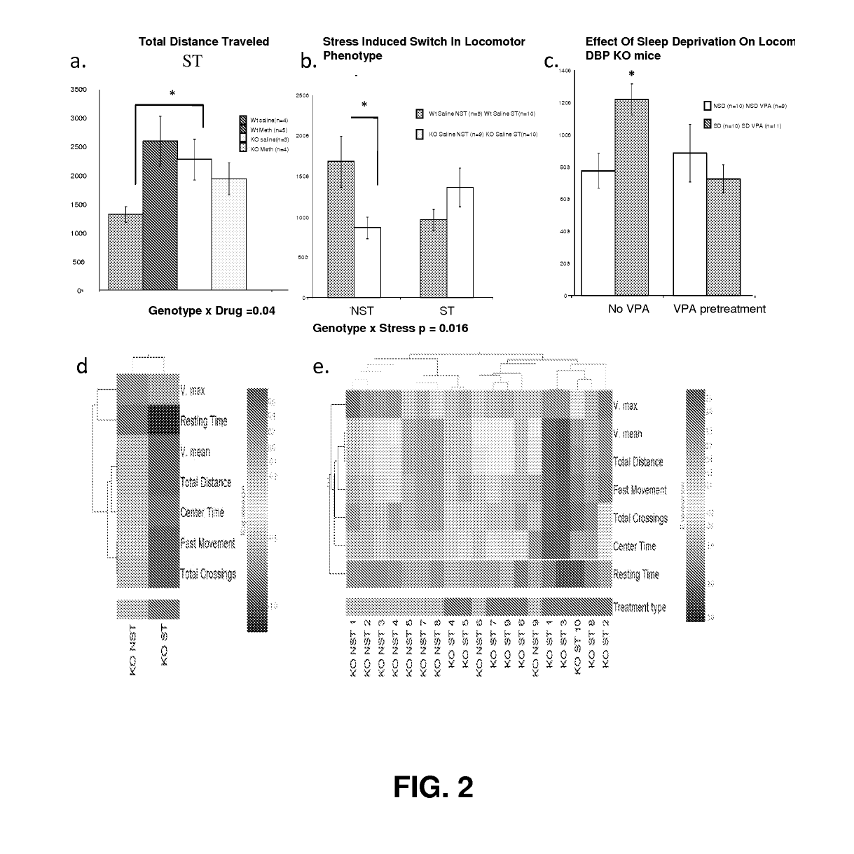

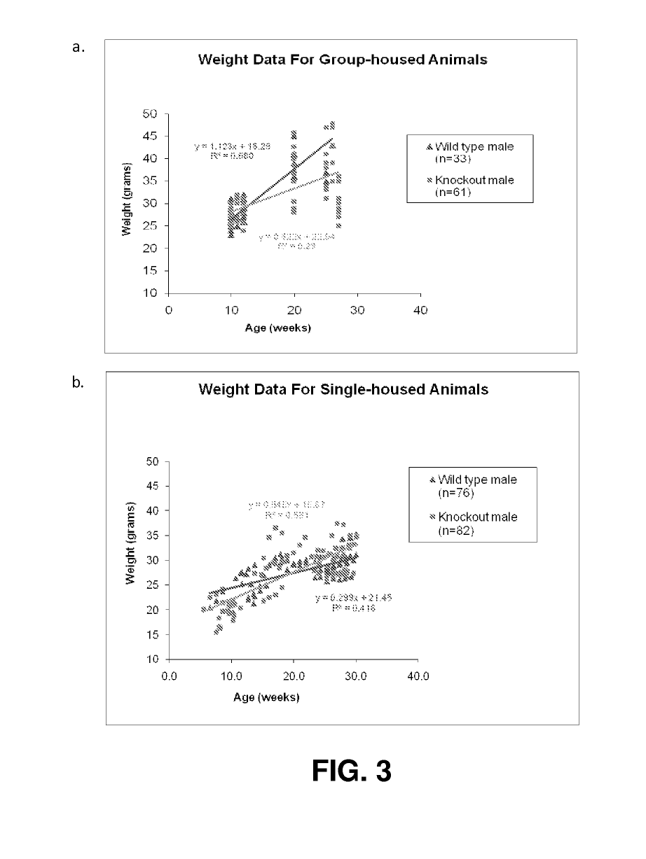

Acute overwhelming stress (accidents, illness, loss of employment) on top of the chronic stress of social isolation often precede decompensation in human bipolar patients. The mice were subjected to a chronic stress paradigm consisting of isolation (single housing) for one month, overlaid with an acute stressor (a series of behavioral challenge tests) at the end of the third week of isolation. When subjected to the chronic stress (ST) paradigm prior to the locomotor assessment, DBP KO ST mice display a change in their locomotor phenotype, becoming hyper-locomotive, while wild type animals become hypolocomotive (FIG. 2b). This switch from a low level of locomotion to a high level of locomotion is analogous to the switch from a depressed phase to an activated (manic) phase of bipolar disorder, and to the activation triggered by stress in Post-Traumatic Stress Disorder (PTSD). Notably, there is a high rate of co-morbidity between PTSD and bipolar disorder. Bipolar mood disorders and stress are often associated clinically with increased alcohol consumption and frank alcoholism.

An unsupervised two-way hierarchical clustering of the mouse locomotor behavioral data measures (phenes) (FIGS. 2d and 2 e) using GeneSpring is illustrative in terms of the bipolar-like phenomenology and the switch from a depression-like state to a mania-like state in response to stress. The NST DBP KO mice and the ST DBP KO mice for the most part clustered into two distinct groups, illustrating the utility of the phenotypic battery of measures in distinguishing between the two groups (FIG. 2e). The heat-plot shows that the phene that was most different in ST mice (decreased) and NST mice (increased) was Resting Time, which has strong analogies to behavioral correlates of mood in humans. Center Time (time spent in the center quadrant of the open field), was increased in ST mice compared to NST mice. Increased Center Time may be a reflection of expansive, exploratory and risk-taking behavior, as mice tend to avoid the potentially dangerous center area of an open-field due to ancestral self-preservation mechanisms. This result illustrates the power of an unbiased approach in identifying simple putative mouse behavioral correlates of mood.

To further characterize the behavioral phenotype of the DBP KO mice, group-housed (NST) male DBP KO mice were subjected to sleep deprivation for a 24 hour period. Following sleep deprivation, sleep-deprived (SD) mice and control non sleep-deprived (NSD) mice were monitored with video-tracking software. SD DBP KO animals displayed a significant increase in the total distance traveled compared to the NSD animals (FIG. 2c). In a second sleep deprivation experiment, mice were pre-treated with an IP valproate injection (200 mg/kg) immediately prior to the sleep deprivation experiment. If the change in locomotor behavior that accompanies sleep-deprivation in the SD animals is representative of an endophenotype that is associated with bipolar disorder, then administration of the mood stabilizing agent valproate should counteract the behavioral response of DBP KO mice to sleep deprivation. Indeed, when valproate was administered prior to sleep deprivation there was no significant difference in the locomotor behavior of SD and NSD animals (FIG. 2c). Of note, valproate treatment did not have any significant effect on locomotion in NSD animals, as the NSD valproate treated animals displayed locomotion that was comparable to the NSD non-valproate treated animals.

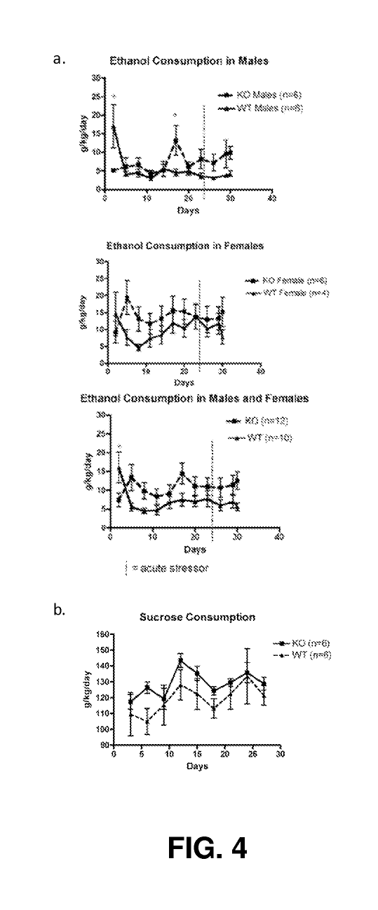

The consumption of alcohol by DBP KO mice was studied. DBP is increased in alcohol preferring (P) rats vs. alcohol non-preferring (NP) rats in the PFC, which indicates that the hypothesis that lower levels or absence of DBP, such as in DBP KO mice, might be associated with decreased consumption of alcohol. However, this may only be applicable to DBP KO mice that are not stressed (NST), and are displaying a depressive-like phenotype. Conversely, ST DBP KO mice that exhibit an activated, manic-like behavior may display an elevated propensity to abuse hedonic substances (alcohol, sucrose) compared to wild type controls. Indeed, while the NST DBP KO mice consume at baseline less alcohol than WT mice, they exhibited a switch in response to stress: ST DBP KO mice consumed more alcohol over a 30 day period as compared to ST wild type mice (FIG. 4a). There was a similar trend in regards to sucrose consumption (FIG. 4b). This evidence strongly indicates that DBP KO mice is a useful model for studying alcohol abuse co-morbidity with bipolar disorder, in relationship to the phases of the illness and response to stress.

### Example 2

### Gene Expression Studies and Convergent Functional Genomics

To understand the molecular underpinnings of the observed phenomenology, brain gene expression profiling studies were carried out using microarrays. In order to identify new marker genes for bipolar disorder, alcoholism and stress reactivity, an expanded Convergent Functional Genomics analysis was conducted (FIG. 5). Gene expression studies was extended to blood in order to identify candidate blood biomarkers (Table 6). Blood biomarkers—genes that change in expression in the blood in concordance with brain changes, serve as a useful tool for diagnosis and for monitoring response to treatment.

A Convergent Functional Genomics approach to interpret the data from a Bayesian perspective, assessing each gene's relevance based on animal model and human lines of evidence (FIG. 5). Internal lines of evidence reflect the new information generated by series of experiments: being changed in expression by loss of the DBP gene in two key brain regions (PFC, AMY) and in blood. As external lines of evidence, mouse QTL data, human genetic linkage or association data, human postmortem brain data, and human blood (lymphocyte) data (FIG. 5) were used. Each line of evidence received an empirical score of 1 if it was related to bipolar disorder, alcoholism or stress/anxiety, and 0.5 if it was related to other neuropsychiatric disorders. These external lines of evidence suffer from the obvious drawback of being constrained by what has been published so far, limiting novelty, and to the inherent biases and limitations of those particular lines of work. Moreover, these external criteria are arguably broad, and may benefit from future parsing. Including disorders other than bipolar disorder, alcoholism and/or stress disorder in the external lines of evidence may dilutes the specificity of this approach. Nevertheless it was decided to include them as a way of increasing sensitivity, based on the emerging clinical, neurobiological and genetic evidence of substantial overlap between major neuropsychiatric disorders, as well as the likelihood that published bipolar and alcoholism-related datasets to date are non-exhaustive. Totaling all the internal and external lines of evidence gives a maximum possible score of 6 points, with the animal model evidence and the human evidence weighted equally (FIG. 5).

Although it is possible that some of the candidate genes that have been identified may be false positives due to potential biological or technical limitations of the methodology and approach employed, the higher the number of independent lines of evidence, the lower the likelihood of that being the case. According to Bayesian theory, an optimal estimate results from combining prior information with new evidence. Different ways of scoring the independent lines of evidence can also be used, which may give somewhat different results in terms of the prioritization of the top candidate genes, if not in terms of the actual content of the list per se. A weighted scoring approach, as used herein, is arguably a reasonable compromise between specificity and sensitivity, between focus and broadness.

For the bipolar comparison, the top candidate genes from the pharmacogenomic model (Ppplrlb/Darpp-32, Penk, Tac1, Mef2c, Gpr88) were also changed in the genetic DBP KO model. This is an unexpectedly strong cross-validation between two independent and very different approaches, a genetic animal model and a pharmacogenomic animal model of bipolar disorder. The direction of change in the PFC (decreased in the DBP KO ST mice, increased in the pharmacogenomic model), is consistent with the interpretation of the behavioral data in the two studies—an activated state in the DBP KO ST mice, and a depressed state in the pharmacogenomic model, at the time of gene expression sampling.

Darpp-32, Penk and Tac1 each showed a remarkable opposite direction of change in the PFC and AMY of the DBP KO ST mice, being decreased in the PFC and increased in the AMY. This indicates that they may provide molecular underpinnings for the reciprocal cortical-limbic dysregulation seen in affective disorders by imaging studies. Moreover, Darpp-32 switched from being decreased in AMY in the DBP NST mice to being increased in AMY in the DBP ST mice.

Two other genes that show a flip in expression from NST to ST are Tmod2 and Gas5. Tmod2 is increased in the PFC of DBP KO NST mice and decreased in the PFC of DBP KO ST mice. This is strikingly consistent with previous studies that have shown that mice lacking Tmod2 show enhanced hyperactivity, long-term potentiation, and deficits in learning and memory. Moreover, the opposite direction of change in DBP KO NST and DBP KO ST mice supports the possibility that Tmod2 may be a substrate for the observed behavioral changes induced by stress in the model.

A number of the genes changed in DBP KO mice have also been reported changed in human postmortem brains from subjects with bipolar disorder, depression, alcoholism, as well as other related disorders. This cross-validation, on one hand reinforces the validity of the approach, and on the other hand reduces the likelihood that those particular postmortem findings are methodological or gene-environment interactions artifacts of working with post-mortem human tissue. Moreover, it illustrates at a genetic and neurobiological mechanism level the overlap among major neuropsychiatric disorders.

In particular, a group of glia/myelin related genes are decreased in both DBP KO NST and ST mice, as well as in bipolar disorder (Mbp, Cldn11, Plp1, Mobp), depression (Cnp, Mog, Mal, Plp1), schizophrenia (Mbp, Cldn11, Plp1, Mobp, Cnp, Mal) and alcoholism (Mbp, Plp1, Mobp, Cnp, Mog, Mal) postmortem brains. Mag is decreased in DBP ST mice only, as well in bipolar, depression, schizophrenia and alcohol brains. These data point to the strong validity of the genetic mouse model, and implicate glia/myelin pathology as integral to bipolar and related disorders. The commonality of alterations in glia/myelin genes, namely a decrease in expression, across a spectrum of neuropsychiatric disorders suggests that hypofunction of glia/myelin systems may be a sensitive if not specific common denominator for mental illness, perhaps leading to hypofrontality and disregulated control of mood—similar to a loose switch. This may be the underlying neuroanatomical reason for the switch from a depressed to an activated (manic-like) phase in response to stress in the constitutive knock-out mice. Omega-3 polyunsaturated fatty acids may directly target this glia/myelin abnormality. Omega-3 fatty acids have been reported to be clinically useful in the treatment of both mood and psychotic disorders. Deficits in omega-3 fatty acids have been linked to increased depression and aggression in both animal models and humans.

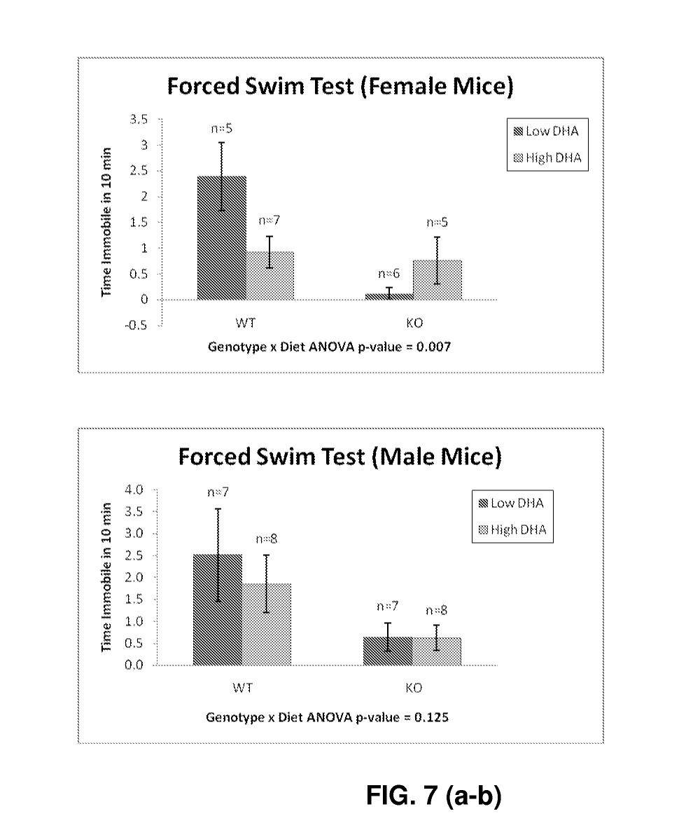

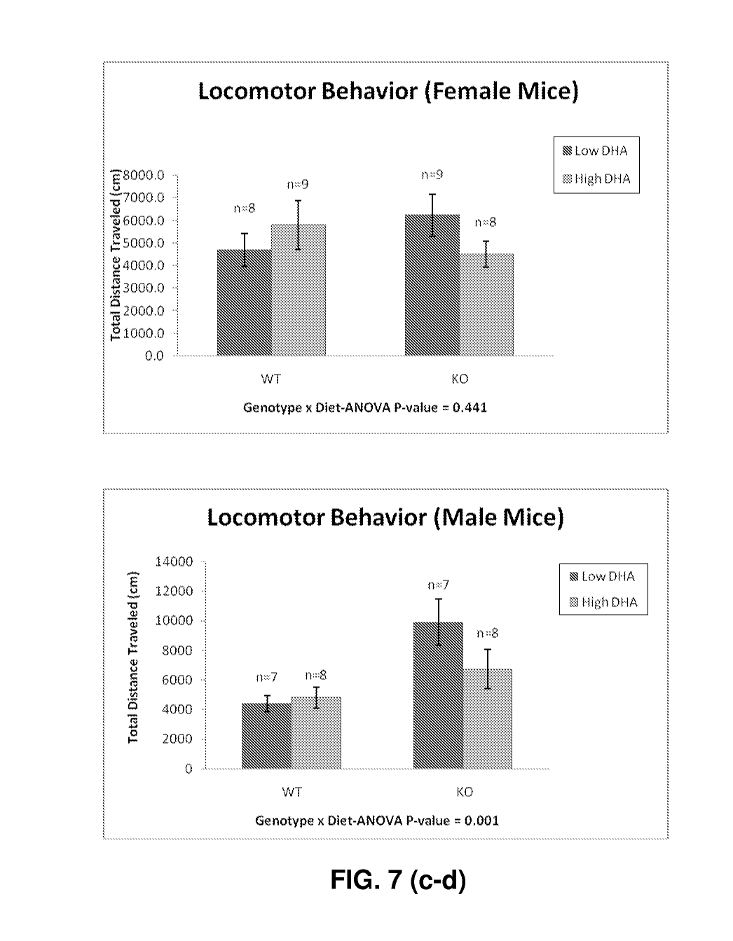

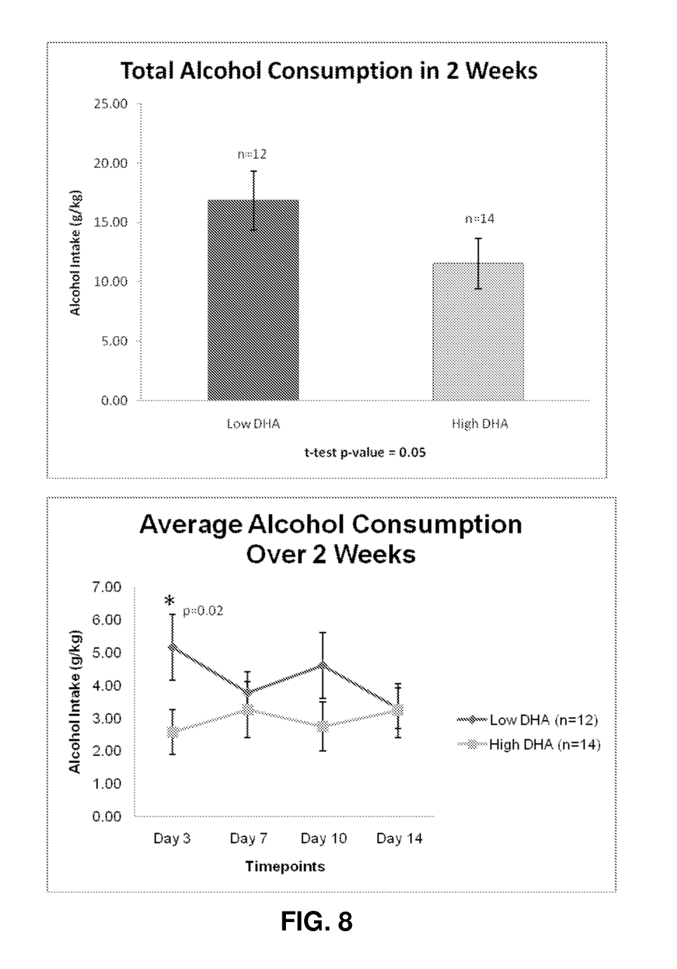

Omega-3 fatty acids have mood modulating properties, in both preclinical models and some small clinical trials. For example, omega-3 fatty acids are clinically useful in the treatment of both mood and psychotic disorders. Deficits in omega-3 fatty acids are linked to increased depression and aggression in both animal models and humans. To date, there is no clear understanding of how they work, or indeed how well they actually work. Unlike most psychiatric drugs, these natural compounds have minimal side-effects, and intriguing evidence for multiple favorable health benefits (cardiovascular, anti-inflammatory, neurodegenerative). Particularly for female patients of child-bearing age, the teratogenic (fetus-harming) side-effects of mood stabilizing medications are a major issue. As such, if the action of omega-3 fatty acids in mood disorders and related disorders are substantiated by understanding their mechanistic effects, they would become an important addition to the therapeutic armamentarium of psychiatrists and primary care doctors. Treatment with the omega-3 fatty acid DHA reverses phenotypic, gene expression and biomarker abnormalities present in DBP KO mouse model (Tables 4-6, FIGS. 7-8). These results demonstrate translational applications for understanding and validating at a molecular level a new treatment approach and may favorably impact multiple co-morbid conditions.

Other interesting examples of genes changed in the animal model for which there is postmortem evidence include Apod, Gsk3b and Ptgs2. Apod (apolipoprotein D) is increased in postmortem brains from bipolar disorder and schizophrenia subjects, and is decreased in brains from depression and alcoholism subjects. In DBP KO ST mice, Apod is increased in the amygdala and decreased in the PFC. Treatment with DHA reverses those changes. Gsk3b (glycogen synthase kinase 3 beta), a target of mood stabilizing drugs, is decreased in postmortem brains from bipolar disorder and depression. In DBP KO ST mice, Gsk3b is increased in the amygdala and decreased in the PFC Ptgs2 (prostaglandin synthase 2) is increased in DBP KO ST mice and in brains from schizophrenia, Alzheimer and multiple sclerosis subjects, suggesting an underlying inflammatory/neurodegenerative phenomenology that may tie in with the glia/myelin hypofunction and the therapeutic effects of omega-3 fatty acids, which also have anti-inflammatory properties. It may be of interest, then, to pursue inhibitors of Ptgs2 (COX2) as therapeutic options in mood disorders with a stress component. Chronic lithium treatment downregulates cyclooxygenase-2 activity in rat brain, and recently the COX2 inhibitor celecoxib was shown to have therapeutic effects in depression in a human clinical trial.

### Example 3

### Stress-Induced Switch in Gene Expression Patterns

The genes changed in opposite directions in the DBP KO NST and DBP KO ST mice (Table 1) are suitable for use as candidate genes and biomarkers for bipolar disorder, as they show a diametric change in conjunction with the switch in phenotype.

PFC: Besides Tmod2, mentioned above, 6 other genes are increased in DBP KO NST mice and decreased in DBP KO ST mice: Kcnb1, Anp32a, Slc1a2, Fut9, Sdc4 and Fundc2. For example, Kcnb1 (voltage-gated potassium channel subunit Kv2.1) regulates neuronal excitability, and has been implicated in protective mechanisms to suppress hyperexcitability. The increase in levels of Kcnb1 we see in the DBP NST mice may underlie neuronal hypoexcitability, and conversely the decrease in levels of KCNB1 in DBP ST mice may underlie neuronal hyperexcitability. This is remarkably congruent with the observed switch in their behavioral phenotype. Slc1a2 (GLT-1/EAAT2, glial high affinity glutamate transporter) is involved in terminating the postsynaptic excitatory actions of glutamate by rapidly removing released glutamate from the synaptic cleft. Increased levels of GLT-1, as in the DBP KO NST mice, would lead to decreased excitability, and decreased levels of GLT-1, as in the DBP KO ST mice, would lead to increased excitability, consistent with the behavioral phenotype observed.

Three genes are decreased in DBP KO NST mice and increased in DBP KO ST mice: Gnb1, Rab39b and Cdh11. For example, Gnb1 (G protein beta 1 subunit gene) is upregulated by psychostimulants and may be involved in the initial behavioral activation response. Consistent with this, it is decreased in DBP KO NST mice, which show reduced locomotion, and increased in DBP KO ST mice, which show increased locomotion. Gnb1 is suppressed by experimental hyperthyroidism in mice, which is intriguing in view of the proposed use of thyroid hormone to treat rapid-cycling bipolar disorder in humans.

A Broad/MIT Connectivity Map analysis of genes that show switch in response to stress in the PFC identified celecoxib, a COX2 inhibitor, as the drug most likely to produce a similar gene expression pattern, and valproate, a mood stabilizer, as one of the drugs most likely to produce an opposite pattern (Table 7). This is an unexpectedly strong independent corroboration of the validity of the genetic animal model, and reinforces the principle of exploring the use of anti-inflammatory agents in the treatment of mood disorders with a stress component, as discussed above.

AMY: Besides Gas5 mentioned earlier, 7 other known genes are switched/decreased by stress: Ap2b1, Eml2, Nup62, Pip5k1b, Rbbp4, Rian, and Sdc4. For example, Pip5k1b (phosphatidylinositol-4-phosphate 5-kinase, type 1 beta) was independently identified as a gene downregulated in response to chronic stress in mice.

Besides Ppp1r1b/Darpp-32 discussed above, 12 other genes are switched/increased by stress: Atp1a1, Gpx3, Irs4, Kcna5, Klhl13, Lhx8, Pbx3, Ptov1, Rasd2, Slc32a1, Vapb, and Zic1. For example, Irs4 (insulin receptor substrate 4) is involved in insulin and fibroblast growth factor receptor signaling. Both the insulin growth factor system and the fibroblast growth factor system have been implicated in the pathogenesis of mood disorders.

Blood: Two genes were switched/decreased by stress: Crisp3 and Klk1b16. These two genes have no known brain functions to date, but may be interesting candidate blood biomarkers of response to stress and switching in bipolar disorder.

### Example 4

### Genes in GeneOntology (Go) Categories that Move Up in the Ranking Following Stress: Sexual Reproduction

A comparison between the biological role categories of DBP NST KO vs. DBP ST KO mice revealed that the GO category of genes related to stress, behavior, and response to stimuli showed the most relative increase in prominence following stress, compared to other biological categories (Table 8 a, b). This demonstrates concordance between molecular changes and behavioral data.

### Example 5

### Candidate Genes and Biomarkers

Clk1 and Drd2 are part of a subset of candidate genes for bipolar/depression identified by CFG analysis in DBP KO NST mice (FIG. 6a). Clk1 (cdc2-like kinase 1) was increased in the DBP KO NST mice, and decreased in brain of mice exposed to psycho-physiological stress. It was also decreased in lymphocytes from schizophrenia patients. Drd2 (dopamine receptor 2) was decreased in the DBP KO NST mice in the AMY, which may be consistent with a depressed state, and was decreased in expression in DBP KO ST mice in the PFC, which may be consistent with an activated, hyperdopaminergic state. It was also decreased in lymphocytes from schizophrenia patients. Some of the other biomarkers for bipolar/depression from the DBP KO NST mice include Itgav, Gls, Enah, Pctk1, Lp1, Gnb1, Kcnj4, Cnp, Hnrpdl, Ywhaz, Clic4, Sgk and Slc38a2 (FIG. 6, Tables 4 and 6). Ywhaz (14-3-3 zeta) maps to a locus on chromosome 8q22.3 that has been implicated in autism, as well as shows some association with schizophrenia. Ywhaz has been reported increased in the PFC of subjects with bipolar disorder, consistent with the increase seen in DBP KO NST mice in brain (PFC, AMY) and blood. Clic4 (chloride intracellular channel 4), a mitochondrial gene, maps to a locus on chromosome 1p36.11 that has been implicated in bipolar disorder and schizophrenia. A decrease in expression of Clic4 was seen in brains of DBP KO NST mice. A decrease in Sgk expression was seen in brain and blood of DBP KO NST mice (Tables 4 and 6), thus it is also a suitable blood biomarker. Sgk expression increased in the AMY of the activated, DBK KO ST mice. Sgk has also been implicated in neuronal plasticity and long-term memory formation in rats. Memory problems are a common clinical feature of depression in humans.

Some of the candidate genes/biomarkers for bipolar/activation identified by the CFG analysis in DBP KO ST mice (FIG. 6b) were Snca and Rxrg. Both were decreased in DBP KO ST mice (FIG. 6 and Table 5), indicating that they may play a protective role against activation. Snca (synuclein alpha) is an abundant and conserved pre-synaptic brain protein, implicated as a critical factor in several neurodegenerative diseases. Snca is decreased in both the brain (amygdala) and blood of DBP KO ST mice, thus being a suitable biomarker. The decreased levels of Snca seen in brain and blood in the mouse model, co-directional with the decrease reported in postmortem brains from alcoholics, indicates that it may have a protective role against alcoholism, and thus useful as potential biomarkers for alcoholism, cravings and risk of relapse. The data on Snca is a remarkable example of translational convergence, and an unexpectedly strong validation of the relevance of the animal model. Treatment with DHA reverses those changes. Rxrg (retinoid X receptor, gamma), a nuclear receptor member of the retinoid signaling pathway, has been implicated in circadian and seasonal changes in energy metabolism and body weight. Rxrg knock-out mice have been reported to have thyroid hormone resistance and increased metabolic rate. This may be consistent with the finding of decreased Rxrg in the PFC of the activated DBP KO ST mice (Table 5).

Some of the other novel candidates genes/biomarkers for bipolar/activation from the DBP KO ST mice include Sfpgm, Hspa1a, Fos, Mal, Drd2, Jak1, Egr1, Gnb1, and Lp1.

An interrogation of the candidate genes/biomarkers from NST and ST mice, for classification in functional groups that had been implicated or hypothesized to have relevance to the pathophysiology of bipolar and related disorders, yielded genes related to glia/myelin function, GABA, glutamate, dopamine, circadian clocks, G-protein coupled receptors, signal transduction, transcription factors, neuropeptides, synaptic function, transporters, ion channels, and neuronal migration/neurite growth.

Data show gene expression changes in two key dopamine receptor genes. Drd1 and Drd2 are both decreased in the PFC of DBP KO ST mice. Human genetic association studies and postmortem work support a direct role of Drd1, and to a lesser extent Drd2, in bipolar disorder. The receptor downregulation, together with their hyperlocomotor phenotype, indicates that these mice may have chronic elevated extracellular dopamine levels, a likely feature of elevated mood states/mania.

In addition to DBP, which was constitutively knocked-out, and Rxrg mentioned above, four other clock-related genes, Csnk1e, Tef, Rora and Rorb were found to be changed in DBP KO mice. Csnk1e (casein kinase 1, epsilon) is a core component of the circadian clock. Animal models and human genetic association studies suggest that Csnk1e contributes to variability in stimulant (amphetamine) response. Interestingly, Csnk1e is a key component in the Darpp-32 (Dopamine-And-cAMP-Regulated-Phosphoprotein-32 kDa) second messenger pathway. Tef (thyrotrophic embryonic factor) is a transcription factor from the same PAR bZip family as DBP. It binds to and transactivates the Tshb promoter and Bnp promoter, among others—regulating thyroid hormone levels and fluid-electrolyte levels respectively. Both these activities are related to level of energy and physiological tonus. Consistent with this, Tef is decreased in PFC of DBP KO NST mice, which show a depression-like phenotype. Perhaps consistent with the increased excitability and reactivity to stress of the DBP KO mice, mice deficient for multiple PAR bZip proteins are highly susceptible to generalized spontaneous and audiogenic epilepsies. Both Rorb (RAR-related orphan receptor B) and Rora (RAR-related orphan receptor A) were increased in AMY and decreased in PFC in DBP KO NST mice. Perhaps consistent with the gene expression results and behavioral data, Rora sg/sg mutant mice, which lack Rora activity, exhibit an enhanced response to novel environment stress, mediated through corticosterone circadian rhythm abnormalities. Of note, corticosterone abnormalities are prominent clinical findings in human affective disorders patients.

A number of potassium channel genes, such as Kcnb1, Kcnj10, Kcnv1 and others are changed in the DBP KO mice. Potassium channels are modulated by anti-epileptic drugs, which are a mainstay of treatment in mood disorders. Kcnj10 had decreased in expression in both DBPKO NST and DBP KO ST mice. The findings of decreases in glia/myelin related genes discussed above, the results are consistent with an overall glia hypofunction in DBP KO mice, in concordance with findings in human mood disorders and alcoholism patients.

### Materials and Methods

### Mouse Colony:

A transgenic mice carrying DBP-KO was generated. The 129/Ola DBP mice, carrying a null allele for the DBP gene, were received from the Schibler group (University of Geneva, Switzerland). The mice were re-derived on a C57/BL6 background at the UCSD Transgenic Mouse and Gene Targeting Core. Mice were subsequently maintained on this mixed background by heterozygote breeding, as described below, and not further back-crossed to C57/BL6. Storage and breeding of the mice took place at the San Diego VA Medical Center and subsequently at the Indiana University School of Medicine in Association for Assessment and Accreditation of Laboratory Animal Care-approved animal facilities, which met all state and federal requirements for animal care.

DBP (+/−) heterozygous (HET) mice were bred to obtain mixed littermate cohorts of wild-type (+/+) (WT), HET and DBP (−/−) knock-out (KO) mice. Mouse pups were weaned at 21 days and housed in groups of two to four (segregated by sex), in a temperature- and light-controlled colony on reverse cycle (lights on at 2200 h, lights off at 1000 h), with food and water available ad libitum. DNA for genotyping was extracted by tail digestion with a Qiagen Dneasy Tissue kit, following the protocol for animal tissue (Qiagen, Valencia, Calif.). We used the following three primers for genotyping by PCR:

Dbp forward: TTCTTTGGGCTTGCTGTTTCCCTGCAGA
Dbp reverse: GCAAAGCTCCTTTCTTTGCGAGAAGTGC
(WT allele)
lacZ reverse: GTGCTGCAAGGCGATTAAGTTGGGTAAC
(KO allele)

Only WT and KO's were used for experiments. Behavioral and gene expression experiments were carried out with mice 8-12 weeks of age.

Animal Housing: All mice were housed for at least two weeks prior to each experiment in a room set to an alternating light cycle with 12 hours of darkness from 10 a.m. to 10 p.m., and 12 hours of light from 10 p.m. to 10 a.m.

Drugs: Mice were administered saline, valproate (200 mg/kg), or methamphetamine (10 mg/kg) acutely by intra-peritoneal injection.

Locomotor Pattern Testing: A SMART II Video Tracker (VT) system (San Diego Instruments, San Diego, Calif.) was used to track movement of mice immediately after drug administration and again 24 hours later. After injection, mice were placed in the lower-right-hand corner of one of four adjacent, 41×41×34-cm³ enclosures. Mice had no physical contact with other mice during testing. Each enclosure has nine pre-defined areas, i.e. center area, corner area, and wall area. The movements of the mice were recorded for 30 minutes.

Measures of overall locomotor activity were obtained and represented by the total distance traveled within and between each of the nine regions of the enclosure. Two categories of behavior were obtained. First, the amount of locomotor activity was assessed by using the total distance traveled in the open field in a 30-minute interval. Second, the spatial scaling exponent, d, or spatial D, was obtained. Spatial D is a quantified measure of the geometric patterns of locomotor activity. Briefly, spatial d is a measure of the non-linear nature of an animal's locomotor movement and is quantified on a scale from 1 to 2; with d=1 indicating extremely linear movement and d=2 representing highly non-linear locomotor movement.

Data and Statistical Analysis: Two-way analyses of variance (ANOVAs) were used to compare total distance traveled and spatial d. Genotype and/or drug treatment were between-subjects variables, and time was a within-subjects variable. All computations were conducted with SPSS statistical software (SPSS Inc., Chicago, Ill.).

Non-Stress (NST) vs. Stress (ST) Experiments: For the Non-Stress (NST) experiments, mice were group housed. For the Stress (ST) experiments, mice were subjected to a chronic stress paradigm consisting of isolation (single housing) for one month, with an acute stressor (behavioral challenge tests) in Week 3. The behavioral challenge tests consisted of sequential administration of the forced swim test, tail flick test and tail suspension test (data not shown). At four weeks, mice were injected with either saline or methamphetamine. Locomotor activity was measured immediately after drug administration and again 24 hours later, immediately after which the brains were harvested for microarray studies.

Sleep Deprivation Experiments: Sleep deprivation studies consisted of light cycle changes, with no handling of animals involved, to avoid inducing non-sleep related handling stress confounds. Male DBP KO mice were used in the sleep deprivation experiments as follows: sleep deprived (SD) animals were removed from the standard housing room with a 12 hour off/12 hour on (reverse) light cycle and kept in a dark room overnight the night before the experiment. Non-sleep deprived (NSD) animals were kept in the housing room with the standard light cycle the night before the experiment to allow for a normal night's sleep. On the day of the experiment, mice were injected with saline (to keep conditions comparable to all of the other behavioral experiments) and locomotor activity was measured immediately afterward with video tracking software. Following the video tracking experiment, animals were sacrificed and the blood of each individual mouse was collected for future biomarker microarray studies. In another series of experiments, sleep deprivation studies were performed as described above with the addition of a valproic acid injection (200 mg/kg) to both the SD and NSD animals 24 hours before videotracking.

Alcohol Consumption Experiments: To create an alcohol free-choice drinking padadigm, both male and female, wild type and DBP KO mice were placed in individual cages with both a bottle of water and a bottle of 10% ethanol. Fluid consumption from both bottles was monitored for a period of 30 days with an acute stressor (as described in Non-Stressed vs. Stressed Experiments above) at the end of the third week. To determine consumption, the weight of each bottle was recorded every three days, at which time the place of the two bottles in each cage was switched. Following thirty days of free-choice drinking the animals were injected with saline and their locomotor activity was assessed with videotracking software. After videotracking we harvested the brain and the blood of each animal for use in future microarray studies.

Clustering analysis of behavioral data: GeneSpring version 7.2 was used (Agilent Technologies, Palo Alto, Calif.). Unsupervised two-way hierarchical clustering of normalized (Z-scored) behavioral data from open-field video-tracking was carried out, using known methodology. Cohen's d effect size was used to standardize the locomotor behavior data for both non-stressed and stressed DBP KO mice: M₁−M₂/σ_{pooled} (M₁ is the average value from the designated DBP KO group for the locomotor measurement of interest. M₂ is the average value form the wild type group for that same locomotor measurement, and σ_{pooled} is the standard deviation of all the values that went into calculating both M₁ and M₂) Clustering of standardized scores was performed with GeneSpring 7.2 software (FIG. 2d). To do a clustering of the scores for individual subjects, we calculated a modified Z score, in which Z score=X₁−M₂/σ_{pooled} (X₁ is the individual score for the locomotor measure of interest, M₂ is the average value form the wild type group for that same locomotor measurement, and σ_{pooled} is the standard deviation of all the values that went into calculating both M₁ and M₂) (FIG. 2e).

RNA extraction and microarray work: Following the 24 hour time-point behavioral test, mice were sacrificed by cervical dislocation. Behavioral testing and tissue harvesting were done at the same time of day in all experiments described in this paper. The brains of the mice were harvested and stereotactically sliced to isolate anatomic regions of interest. Tissue was flash frozen in liquid nitrogen and stored at −80° C. pending RNA extraction. Approximately 1 ml of blood/mouse was collected into a PAXgene blood RNA collection tubes, BD diagnostic (VWR.com). The Paxgene blood vials were stored in −4° C. overnight, and then at −80° C. until future processing for RNA extraction.

Standard techniques were used to obtain total RNA (22 gauge syringe homogenization in RLT buffer) and to purify the RNA (RNeasy mini kit, Qiagen, Valencia, Calif.) from micro-dissected mouse brain regions. For the whole mouse blood RNA extraction, PAXgene blood RNA extraction kit (PreAnalytiX, a QIAGEN/BD company) was used, followed by GLOBINclear™-Human or GLOBINclear™-Mouse/Rat (Ambion/Applied Biosystems Inc., Austin, Tex.) to remove the globin mRNA. All the methods and procedures were carried out as per manufacturer's instructions. The quality of the total RNA was confirmed using an Agilent 2100 Bioanalyzer (Agilent Technologies, Palo Alto, Calif.). The quantity and quality of total RNA was also independently assessed by 260 nm UV absorption and by 260/280 ratios, respectively (Nanodrop spectrophotometer). Starting material of total RNA labeling reactions was kept consistent within each independent microarray experiment.

For brain, equal amounts of total RNA extracted from brain tissue samples (pre-frontal cortex, amygadala) from individual mice was used for labeling and hybridization to Mouse Genome 430 2.0 arrays (Affymetrix, Santa Clara, Calif.). For blood, material from 3 mice was pooled for each experimental condition. The GeneChip Mouse Genome 430 2.0 Array contain over 45,000 probe sets that analyze the expression level of over 39,000 transcripts and variants from over 34,000 well-characterized mouse genes. Standard Affymetrix protocols were used to reverse transcribe the messenger RNA and generate biotinlylate cRNA (Affymetrix, Inc., CA).

The amount of cRNA used to prepare the hybridization cocktail was kept constant within each experiment. Samples were hybridized at 45° C. for 17 hours under constant rotation. Arrays were washed and stained using the Affymetrix Fluidics Station 400 and scanned using the Affymetrix Model 3000 Scanner controlled by GCOS software. All sample labeling, hybridization, staining and scanning procedures were carried out as per manufacturer's recommendations.

Quality control: All arrays were scaled to a target intensity of 1000 using Affymetrix MASv 5.0 array analysis software. Quality control measures including 3′/5′ ratios for GAPDH and beta-actin, scaling factors, background, and Q values were within acceptable limits.

Microarray data analysis: Data analysis was performed using Affymetrix Microarray Suite 5.0 software (MAS v5.0). Default settings were used to define transcripts as present (P), marginal (M), or absent (A). For brain, a comparison analysis was performed for individual KO saline mouse, using individual WT saline mice as the baseline. “Signal,” “Detection,” “Signal Log Ratio,” “Change,” and “Change p-value,” were obtained from this analysis. Only transcripts that were called Present in at least one of the two samples in a comparison pair, and that were reproducibly changed in the same direction in at least 6 out of 9 comparisons, were analyzed further. For blood, a comparison analysis was performed for pooled KO saline mice blood, using pooled WT saline mice blood as the baseline. “Signal,” “Detection,” “Signal Log Ratio,” “Change,” and “Change p-value,” were obtained from this analysis. Only transcripts that were called Present in at least one of the two samples in a comparison pair, and that were reproducibly changed in the same direction in two independent biological experiments, were analyzed further.

Gene identification: The identities of transcripts were established using NetAFFX (Affymetrix, Santa Clara, Calif.), and confirmed by cross-checking the target mRNA sequences that had been used for probe design in the Affymetrix Mouse Genome 430 2.0 arrays GeneChip® with the GenBank database. Where possible, identities of ESTs were established by BLAST searches of the nucleotide database. A National Center for Biotechnology Information (NCBI) (Bethesda, Md.) BLAST analysis of the accession number of each probe-set was done to identify each gene name. BLAST analysis identified the closest known mouse gene existing in the database (the highest known mouse gene at the top of the BLAST list of homologues) which then could be used to search the GeneCards database (Weizmann Institute, Rehovot, Israel) to identify the human homologue. Probe-sets that did not have a known gene were labeled “EST” and their accession numbers kept as identifiers.

Human Genetic Linkage Convergence: To designate convergence for a particular gene, the gene had to map within 10 cM of a microsatellite marker for which at least one published study showed evidence for linkage to bipolar, alcoholism, or other co-morbid neuropsychiatric disorders (depression, stress, anxiety). The University of Southampton's sequence-based integrated map of the human genome (The Genetic Epidemiological Group, Human Genetics Division, University of Southampton) was used to obtain cM locations for both genes and markers. The sex-averaged cM value was calculated and used to determine convergence to a particular marker. For markers that were not present in the Southampton database, the Marshfield database (Center for Medical Genetics, Marshfield, Wis., USA) was used with the NCBI Map Viewer web-site to evaluate linkage convergence.

Mouse QTL Convergence: To search for mouse QTL (Quantitative Trait Loci) evidence for candidate genes, the MGI_{—}3.54—Mouse Genome Informatics (Jackson Laboratory), the search menu for mouse phenotypes and mouse models of human disease phenotype ontology were used searching for abnormal behaviors related to depression, alcoholism, fear/anxiety. To designate convergence for a particular gene, the gene had to map within 10 cM of a QTL marker for the abnormal behavior.

Human Tissue (Postmortem Brain,Blood) convergence: Information about candidate genes was obtained using GeneCards, the Online Mendelian Inheritance of Man database, as well as database searches using PubMed and various combinations of keywords (gene name, bipolar, depression, alcoholism, stress, anxiety, human, postmortem, brain, blood).

Gene Ontology (GO) analysis: The NetAffx Gene Ontology Mining Tool (Affymetrix, Santa Clara, Calif.) was employed to categorize the genes in the datasets into functional categories, using the Biological Process ontology branch.

Ingenuity Pathway Analysis: Ingenuity 5.1 (Ingenuity Systems, Redwood City, Calif.) was employed to identify genes in the datasets that are the target of existing drugs, as well as used to analyze the direct interactions of candidate genes/biomarkers resulting from the CFG analysis.

DHA (omega-3 fatty acids) experiments: Male and female DBP WT (+/+) or DBP(−/−) KO mice were placed on one of 2 diets:

(1) Low Omega-3 fatty acid custom research diet (TD. 00522 Harlan Teklad, Madison, Wis.) a DHA-depleting low n−3 PUFA test diet adequate in all other nutrients [n−6/n−3 ratio of 85:1 with 6% fat as safflower oil] (Lim et al., 2005); or (2) a High Omega-3 custom research diet (TD. 07708 low-DHA diet supplemented with 0.69% DHA (Martek Bioscience, Columbia, Md.) (Lim et al., 2005). The DBP mice were fed low-DHA chow (0% DHA), or high-DHA chow (0.69% DHA) for 28 days. Mice, food, and water were weighed twice a week. Water was refilled once a week. During the 28 day period, the mice were single-housed to induce chronic stress, and underwent behavioral challenge tests on day 21 of the experiment to induce acute stress. The behavioral challenge tests consisted of sequential administration of the forced swim test, tail flick test, and tail suspension test. At four weeks (day 28) the mice were injected with saline and their locomotor activity was assessed with videotracking software. After videotracking the brain and blood of each animal were harvested [microsurgery to separate brain into regions] for use in microarray studies.

Alcohol Studies with DHA: To create an alcohol free-choice drinking padadigm, male WT and DBP KO mice were placed in individual cages with both a bottle of ˜250 ml cold tap water and a bottle of ˜250 ml 10% ethanol, and either a low or high DHA diet for 14 days. The amount of ethanol and water consumed was recorded twice a week, at which time the location of the bottles were switched to prevent positional bias. The bottles were refilled with fresh solution once a week.

TABLE 1
Genes changed in DBP KO mice

| | | | Switched-Changed in |
|---|---|---|---|
| | | | opposite directions in NS [symbol] |
| | DBP KO saline | DBP KO | Gene symbol, direction |
| Region | Non Stressed | saline Stressed | of change NST, ST |
| | | | Switched/Increased by stress: |
| PFC | 65 Decreased | 325 Decreased | Gnb1 (D, I) |
| | 34 Increased | 102 Increased | Cdh11/2610005L07Rik /// LOC546041 (D, I) |
| | | | Rab39b (D, I) |
| | | | Switched/Decreased by stress: |
| | | | Anp32a (I, D) |
| | | | Fundc2 (I, D) |
| | | | Fut9 (I, D) |
| | | | Kcnb1 (I, D) |
| | | | Sdc4 (I, D) |
| | | | Tmod2 (I, D) |
| | | | Slc1a2 (I, D) |
| | | | Switched/Increased by stress: |
| AMY | 228 Decreased | 147 Decreased | Atp1a1 (D, I) |
| | 206 Increased | 177 Increased | Gpx3 (D, I) |
| | | | Irs4 (D, I) |
| | | | Kcna5 (D, I) |
| | | | Klhl13 (D, I) |
| | | | Lhx8 (D, I) |
| | | | LOC669637 (D, I) |
| | | | Pbx3 (D, I) |
| | | | Ppp1r1b (D, I) |
| | | | Ptov1 (D, I) |
| | | | Rasd2 (D, I) |
| | | | Slc32a1 (D, I) |
| | | | Vapb (D, I) |
| | | | Zic1 (D, I) |
| | | | Switched/Decreased by |
| | | | stress: |
| | | | Ap2b1 (I, D) |
| | | | C230078M08Rik (I, D) |
| | | | Eml2 (I, D) |
| | | | Gas5 (I, D) |
| | | | Nup62 (I, D) |
| | | | Pip5k1b (I, D) |
| | | | Rbbp4 (I, D) |
| | | | Rian (I, D) |
| | | | Sdc4 (I, D) |
| | | | Switched/Decreased by stress: |
| Blood | 136 Decreased | 28 Decreased | Crisp3 (I, D) |
| | 9 Increased | 3 Increased | Klk1b16 (I, D) |

[symbol]

indicates data missing or illegible when filed

TABLE 2
Overlap with bipolar pharmacogenomic model CFG analysis.

| Entrez | | | | Bipolar CFG |
|---|---|---|---|---|
| ID | Gene Symbol - Gene Name | DBP NST | DBP ST | (Ogden et al. 2004) |
| 84152 | Ppp1r1b/Darp32 | AMY - D | AMY - I | PFC Cat I-Meth(I) VPA(I) |
| | protein phosphatase 1, regulatory (inhibitor) | PFC - D | PFC - D | |
| | subunit 1B | | | |
| 53635 | Ptov1 | AMY - I | AMY - I | AMY Cat III-VPA(I) |
| | prostate tumor overexpressed gene 1 | | | |
| 2782 | Gnb1 | AMY - D | PFC - I | AMY Cat IV-Meth (I) |
| | guanine nucleotide binding protein (G protein), beta | PFC - D | | CP Cat IV-VPA (D) |
| | polypeptide 1) | | | |
| 60674 | Gas5 | AMY - I | AMY - D | AMY Cat IV-Meth(I) |
| | growth arrest-specific 5 | | | |
| 60 | Actb | AMY - D | PFC - D | AMY Cat IV-VPA (I) |
| | actin, beta | | | PFC Cat IV-Meth (D) |
| 29767 | Tmod2 | PFC - I | PFC - D | PFC Cat IV-METH (D) |
| | tropomodulin 2 | | | |
| 4205 | Mef2a | AMY - D | PFC - D | AMY Cat IV-Meth (D) |
| | MADS box transcription enhancer factor 2, | PFC - D | | CP Cat IV-VPA (I) |
| | polypeptide A (myocyte enhancer factor 2A) | | | |
| 84867 | Ptpn5/Step | AMY - D | PFC - D | AMY Cat IV-VPA (I) |
| | protein tyrosine phosphatase, non-receptor type 5 | | | |
| 7077 | Timp2 | AMY - D | PFC - D | AMY Cat IV-VPA (D) |
| | tissue inhibitor of metalloproteinase 2 | | | |
| 5179 | Penk1 | | PFC - D | PFC Cat I-Meth(I) VPA(I) |
| | preproenkephalin | | AMY - I | |
| 6863 | Tac1 | | AMY - I | PFC Cat I-Meth(I) VPA(I) |
| | tachykinin, precursor 1 (substance P) | | PFC - D | |
| 54112 | Gpr88 | | PFC - D | PFC Cat I-Meth(I) VPA (I) |
| | G-protein coupled receptor 88 | | | |
| 4208 | Mef2c | | AMY - I | PFC Cat I-Meth(D) VPA(I) |
| | myocyte enhancer factor 2C | | | AMY Cat III-VPA(D) |
| 1159 | Ckmt1 | | AMY - D | AMY Cat III-Meth(I) VPA(I) |
| | creatine kinase, mitochondrial 1 (ubiquitous) | | | |
| 9871 | Sec24d | | AMY - D | AMY Cat III-Meth(I) |
| | SEC24 related gene family, member D (S. cerevisiae) | | | |
| 214 | Alcam | | AMY - I | AMY Cat IV-VPA(D) |
| | activated leukocyte cell adhesion molecule | | PFC - D | PFC Cat III-Meth(D) |
| | | | | VT Cat IV-Meth(D) |
| | | | | CP Cat IV-VPA(D) |
| 2932 | Gsk3b | | PFC - D | PFC Cat IV-METH (D) |
| | glycogen synthase kinase 3 beta | | AMY - I | CP IV-VPA (D) |
| 2788 | Gng7 | | PFC - D | PFC Cat III-Meth(I) VPA(I) |
| | guanine nucleotide binding protein (G protein), | | | |
| | gamma 7 subunit | | | |
| 1656 | Ddx6 | | AMY - I | PFC Cat III-Meth (D) |
| | DEAD (Asp-Glu-Ala-Asp) box polypeptide 6 | | PFC - D | VT Cat IV-Meth (D) |
| 6330 | Scn4b | | AMY - I | PFC Cat III-Meth(I) VPA(I) |
| | sodium channel, type IV, beta polypeptide | | PFC - D | |
| 25831 | Hectd1 | | AMY - I | PFC Cat IV-Meth(D) |
| | HECT domain containing 1 | | PFC - D | |
| 5178 | Peg3 | | AMY - I | AMY Cat IV-VPA(D) |
| | paternally expressed 3 | | PFC - D | PFC Cat IV-Meth(D) |
| 394 | Arhgap5 | | PFC - D | PFC Cat IV-Meth (I) |
| | Rho GTPase activating protein 5 | | | |
| 2557 | Gabra4 | | PFC - D | PFC Cat IV-METH (I) |
| | Gamma-aminobutyric acid (GABA-A) receptor, | | | |
| | subunit alpha 4 | | | |
| 5728 | Pten | | AMY - I | PFC Cat IV-Meth(D) |
| | phosphatase and tensin homolog | | PFC - D | |
| 10396 | Atp8a1 | | AMY - I | AMY Cat IV-VPA(D) |
| | ATPase, aminophospholipid transporter (APLT), | | | |
| | Class I, type 8A, member 1 | | | |
| 8851 | Cdk5r1 | AMY - I | | AMY Cat II-Meth(D) VPA(D) |
| | cyclin-dependent kinase 5, regulatory subunit (p35) 1 | | | CP Cat III-VPA(I) |
| 783 | Cacnb2 | AMY - D | | AMY Cat III-VPA(D) |
| | calcium channel, voltage-dependent, beta 2 subunit | | | CP Cat IV-VPA(I) |
| 3423 | Ids | AMY - D | | AMY Cat III-VPA(D) |
| | iduronate 2-sulfatase | | | CP Cat IV-VPA(I) |
| 23122 | Clasp2 | AMY - I | | AMY Cat IV-VPA(D) |
| | CLIP associating protein 2 | PFC - I | | |
| 124925 | Sez6 | AMY - D | | AMY Cat IV-VPA(I) |
| | seizure related gene 6 | | | |
| 7276 | Ttr | AMY - D | | CP Cat IV-Meth(I) |
| | transthyretin | | | |

I—Increased in expression;
D—decreased in expression.
CP—caudate-putamen;
VT—ventral tegmentum.
In bold, genes that show inverse PFC vs. AMY expression.

TABLE 3
Overlap with alcohol CFG analysis

| | | | | Alcohol CFG |
|---|---|---|---|---|
| Entrez | | | | (Rodd, Bertsch et |
| ID | Gene Symbol | DBP NST | DBP ST | al. 2007) |
| 216 | Aldh1a1 | PFC - D | AMY - I | Category IIB- |
| | aldehyde dehydrogenase | | | HIP(I) |
| | family 1, subfamily A1 | | | Category III-1 |
| | | | | PFC(I) |
| 1267 | Cnp | PFC - D | PFC - D | Category III-1 |
| | cyclic nucleotide | | | PFC(I) |
| | phosphodiesterase 1 | | | |
| 4118 | Mal | PFC - D | PFC - D | Category III-1 |
| | myelin and lymphocyte protein, T- | | | PFC(I) |
| | cell differentiation protein | | | |
| 4336 | Mobp | PFC - D | AMY - I | Category III-1 |
| | myelin-associated | | PFC - D | PFC(I) |
| | oligodendrocyte basic protein | | | |
| 4340 | Mog | AMY - D | PFC - D | Category III-1 |
| | myelin oligodendrocyte | PFC - D | | PFC(I) |
| | glycoprotein | | | |
| 5354 | Plp1 | PFC - D | PFC - D | Category III-1 |
| | proteolipid protein (myelin) 1 | | | PFC(I) |
| 1628 | Dbp | PFC - D | AMY - D | Category III-1 |
| | D site albumin promoter binding protein | AMY - D | PFC - D | PFC(I) |
| 3315 | Hspb1 | | PFC - I | Category IIA- |
| | heat shock 27 kDa protein 1 | | | HIP(I) |
| | | | | Category IIB- |
| | | | | PFC(I) |
| 3182 | Hnrpab | | AMY - I | Category IIA- |
| | heterogeneous nuclear | | | AMY(D) |
| | ribonucleoprotein A/B | | | |
| 7086 | Tkt | | PFC - D | Category III-1 |
| | transketolase | | | PFC(I) |
| 6697 | Spr | | PFC - D | Category III-1 |
| | sepiapterin reductase | | | PFC(D) |
| 347 | Apod | | AMY - I | Category III-1 |
| | apolipoprotein D | | PFC - D | PFC(I) |

I—Increased in expression;
D—decreased in expression.
In bold, genes that show inverse PFC vs. AMY expression.

TABLE 4
Top DBP KO NST genes.

| | | Mouse | | | | | | |
|---|---|---|---|---|---|---|---|---|
| | | Brain | | | | | | |
| | | region(s)- | | Mouse | | | | |
| Entrez | Gene Symbol- | Direction | Mouse | Quantitative Trait | Human Genetic | Human Postmortem | Human | [symbol] |
| ID | Gene Description | of change | Blood | Loci (QTL) | Evidence | Brain | Blood | S [symbol] |
| 1813 | Drd2 | AMY-D | | Chr 9 | 11q23.92 | I BP | I SZ | [symbol] |
| | dopamine receptor 2 | | | Abnormal fear/anxiety- | Alcohol | D Depression | | |
| | | | | related behavior, | | D Alcohol | | |
| | | | | Abnormal sleep | | D SZ | | |
| | | | | pattern/circadian | | D Marijuana | | |
| | | | | rhythm | | I Tourette Syndrome | | |
| 1195 | Clk1 | AMY-I | | Chr 1 | 2q33.1 | D Alcohol | D SZ | [symbol] |
| | CDC-like kinase 1 | | | Addiction/drug abuse, | Alcohol | | | |
| | | | | Abnormal fear/anxiety- | SZ | | | |
| | | | | related behavior | Autism | | | |
| 3685 | Itgav | AMY-I | | Chr 2 | 2q32.1 | | I BP | [symbol] |
| | integrin alpha V | | | Abnormal Sleep | BP | | | |
| | | | | pattern/circadian | Alcohol | | | |
| | | | | rhythm | Autism | | | |
| 2744 | Gls | AMY-I | | Chr 1 | 2q32.2 | | D PTSD | [symbol] |
| | glutaminase | | | Abnormal sleep | BP^{[} | | | |
| | | | | pattern/circadian | Alcohol | | | |
| | | | | rhythm, Addiction/ | SZ^{[} | | | |
| | | | | drug abuse, Abnormal | Autism | | | |
| | | | | fear/anxiety-related | | | | |
| | | | | behavior | | | | |
| 7857 | Scg2 | AMY-D | | Chr 1 | 2q36.1 | I SZ | | [symbol] |
| | secretogranin II | | | Anxiety/Fear | Alcohol | D Alcohol | | |
| | | | | | SZ | | | |
| 551 | Avp | AMY-D | | Chr 2 | 20p13 | I Depression | | [symbol] |
| | arginine vasopressin | | | Abnormal fear/anxiety- | BP | | | |
| | | | | related behavior, | | | | |
| | | | | Abnormal sleep | | | | |
| | | | | pattern/circadian | | | | |
| | | | | rhythm, Depression- | | | | |
| | | | | related behavior, | | | | |
| | | | | Addiction/drug abuse | | | | |
| 4842 | Nos1 | AMY-D | | Chr 5 | 12q24.22 | I BP | | [symbol] |
| | Nitric oxide synthase 1, | | | Abnormal sleep | BP | | | |
| | neuronal (Nos1), mRNA | | | pattern/circadian | SZ | | | |
| | | | | rhythm | | | | |
| 6678 | Sparc | AMY-D | | Chr 11 | 5q33.1 | I BP | | [symbol] |
| | secreted acidic cysteine rich | | | Anxiety/Fear (QTL) | BP | Tay-Sachs and | | |
| | glycoprotein | | | | Psychosis^{]} | Sandhoff diseases | | |
| | | | | | Alcohol | | | |
| | | | | | SZ | | | |
| | | | | | Epilepsy^{[} | | | |
| 815 | Camk2a | AMY-I | | Chr 18 | 5q32 | I BP | | [symbol] |
| | calcium/calmodulin- | | | Abnormal Sleep | BP | D BP | | |
| | dependent protein kinase II | | | pattern/circadian | Psychosis | I Depression | | |
| | alpha | | | rhythm | Alcohol^{[} | I SZ^{[} | | |
| | | | | | SZ | | | |
| 4987 | Oprl1 | AMY-D | | Chr 2 | 20q13.33 | I BP | | [symbol] |
| | opioid receptor-like 1 | | | Addiction/drug abuse, | BP | | | |
| | | | | Abnormal fear/anxiety- | SZ | | | |
| | | | | related behavior | Alcohol | | | |
| 7078 | Timp3 | AMY-D | | Chr 10 | 22q12.3 | I Alcohol | | [symbol] |
| | tissue inhibitor of | | | Addiction/drug abuse, | BP | | | |
| | metalloproteinase 3 | | | Abnormal fear/anxiety- | Panic Disorder | | | |
| | | | | related behavior, | | | | |
| | | | | Abnormal sleep | | | | |
| | | | | pattern/circadian | | | | |
| | | | | rhythm | | | | |
| 2562 | Gabrb3 | AMY-D | | Chr 7 | 15q12 | I Alcohol | | [symbol] |
| | gamma-aminobutyric acid | PFC-D | | Addiction/drug abuse | BP | D Epilepsy | | |
| | (GABA-A) receptor, subunit | | | | SZ | D Multiple Sclerosis | | |
| | beta 3 | | | | | | | |
| 4005 | Lmo2 | AMY-D | | Chr 2 | 11p13 | D Alcohol | | [symbol] |
| | LIM domain only 2 | | | Abnormal fear/anxiety- | BP | D SZ^{]} | | |
| | | | | related behavior, | Autism | | | |
| | | | | Depression-related | | | | |
| | | | | behavior | | | | |
| 2571 | Gad1 | AMY-D | | Chr 2 | 2q31.1 | D BP | | [symbol] |
| | glutamic acid decarboxylase 1 | | | Abnormal sleep | BP [symbol] | I SZ | | |
| | | | | pattern/circadian | Alcohol | D Epilepsy | | |
| | | | | rhythm, Depression- | | | | |
| | | | | related behavior, | | | | |
| | | | | Abnormal fear/anxiety- | | | | |
| | | | | related behavior | | | | |
| 1267 | Cnp | PFC-D | D | | 17q21.2 | D Depression | | [symbol] |
| | cyclic nucleotide | | | | Alcohol | D Alcohol | | |
| | phosphodiesterase 1 | | | | Autism^{[} | D SZ | | |
| 324 | Apc | AMY-I | | Chr 18 | 5q22.2 | D BP | | [symbol] |
| | adenomatosis polyposis coli | | | Addiction/drug abuse | Alcohol^{[} | I Alcohol | | |
| | | | | | | I SZ | | |
| 1745 | Dlx1 | AMY-D | | Chr 2 | 2q31.1 | D BP^{[} | | [symbol] |
| | distal-less homeobox 1 | | | Abnormal sleep | BP^{[} | D SZ | | |
| | | | | pattern/circadian | Autism | | | |
| | | | | rhythm, Depression- | | | | |
| | | | | related behavior, | | | | |
| | | | | Abnormal fear/anxiety- | | | | |
| | | | | related behavior | | | | |
| 4340 | Mog | PFC-D | | Chr 17 | 6p22.1 | D BP | | [symbol] |
| | myelin oligodendrocyte | AMY-D | | Anxiety/Fear | BP | D MDD^{[} | | |
| | glycoprotein | | | | Psychosis | D Alcohol^{[} | | |
| | | | | | SZ | D SZ [symbol] | | |
| | | | | | Alcohol | I Epilepsy | | |
| 7881 | Kcnab1 | AMY-D | | Chr 3 | 3q25.31 | D Alcohol | | [symbol] |
| | potassium voltage-gated | | | Abnormal fear/anxiety- | BP | | | |
| | channel, shaker-related | | | related behavior | SZA | | | |
| | subfamily, beta member 1 | | | | Simple Phobia | | | |
| | | | | | Agoraphobia | | | |
| 1282 | Col4a1 | AMY-I | | Chr 8 | 13q34 | D Alcohol | | [symbol] |
| | procollagen, type IV, alpha 1 | | | Abnormal fear/anxiety- | BP [symbol] | | | |
| | | | | related behavior, | | | | |
| | | | | Addiction/drug abuse, | | | | |
| | | | | Depression-related | | | | |
| | | | | behavior | | | | |
| 9987 | Hnrpdl | AMY-I | D | Chr 5 | 4q21.22 | | | [symbol] |
| | heterogeneous nuclear | | | Abnormal sleep | BP | | | |
| | ribonucleoprotein D-like | | | pattern/circadian | Alcohol | | | |
| | | | | rhythm | SZ^{[} | | | |
| 3761 | Kcnj4 | AMY-I | | Chr 15 | 22q13.1 | | I SZ | [symbol] |
| | potassium inwardly- | | | Abnormal fear/anxiety- | BP | | | |
| | rectifying channel, subfamily | | | related behavior | Panic Disorder | | | |
| | J, member 4 | | | Depression-related | | | | |
| | | | | behavior | | | | |
| 2782 | Gnb1 | AMY-D | | Chr 4 | 1p36.33 | I SZ^{[} | I BP | [symbol] |
| | guanine nucleotide binding | PFC-D | | Abnormal fear/anxiety- | | D SZ | | |
| | protein, beta 1 | | | related behavior | | | | |
| 55740 | Enah | AMY-I | | Chr 1 | 1q42.12 | I SZ | | [symbol] |
| | Enabled homolog | | | Abnormal fear/anxiety- | BP | | | |
| | (Drosophila) (Enah), mRNA | | | related behavior | SZ [symbol] | | | |
| | | | | | Autism | | | |
| | | | | | Panic Disorder^{[} | | | |
| 4023 | Lpl | AMY-D | | Chr 8 | 8p21.3 | I SZ | | [symbol] |
| | lipoprotein lipase | | | Abnormal sleep | BP | | | |
| | | | | pattern/circadian | SZ | | | |
| | | | | rhythm, Addiction/ | | | | |
| | | | | drug abuse, | | | | |
| | | | | Depression-related | | | | |
| | | | | behavior | | | | |
| 5127 | Pctk1 | AMY-I | | Chr X | Xp11.3 | I BP | | [symbol] |
| | PCTAIRE-motif protein | | | Abnormal fear/anxiety- | | D SZ | | |
| | kinase 1 | | | related behavior, | | | | |
| | | | | Abnormal sleep | | | | |
| | | | | pattern/circadian | | | | |
| | | | | rhythm, Depression- | | | | |
| | | | | related behavior, | | | | |
| | | | | Addiction/drug abuse | | | | |
| 25932 | Cilc4 | PFC-D | | | 1p36.11 | | I BP^{]} | [symbol] |
| | chloride intracellular | | | | BP | | | |
| | channel 4 (mitochondrial) | | | | SZ | | | |
| 5532 | Ppp3cb | AMY-D | | | 10q22.2 | I SZ | | [symbol] |
| | protein phosphatase 3, | | | | BP | I Alcohol | | |
| | catalytic subunit, beta | | | | Alzheimers | | | |
| | isoform | | | | | | | |
| 7534 | Ywhaz | AMY-I | I | | 8q22.3 | I BP | | [symbol] |
| | tyrosine 3- | PFC-I | | | | D BP | | |
| | monooxygenase/tryptophan | | | | | D SZ | | |
| | 5-monooxygenase | | | | | D Alcohol | | |
| | activation protein, zeta | | | | | | | |
| | polypeptide | | | | | | | |
| 29775 | Card10 | AMY-D | | | 22q13.1 | I Alcohol | | [symbol] |
| | caspase recruitment domain | | | | BP | | | |
| | family, member 10 | | | | Panic Disorder | | | |
| 9783 | Rims3 | AMY-D | | | 1p34.2 | I SZI Alcohol^{]} | | [symbol] |
| | regulating synaptic | | | | SZ^{[} | | | |
| | membrane exocytosis 3 | | | | Anorexia Nervosa | | | |
| 8899 | Prpf4b | AMY-I | | | 6p25.2 | I BP, Major Depression, | | [symbol] |
| | PRP4 pre-mRNA | | | | SZ | SZ | | |
| | processing factor 4 homolog | | | | Alcohol | | | |
| | B (yeast) | | | | | | | |
| 5010 | Cldn11 | PFC-D | | | 3q26.2 | D SZ | | [symbol] |
| | claudin 11 (oligodendrocyte | AMY-D | | | BP^{[} | D BP | | |
| | transmembrane protein) | | | | SZ | I SZ^{[} | | |
| 5168 | Enpp2 | PFC-D | | | 8q24.12 | D MDD | | [symbol] |
| | ectonucleotide | | | | BP | D Alcohol | | |
| | pyrophosphatase/phosphodi | | | | SZA^{[} | I Alcohol | | |
| | esterase 2 (autotaxin) | | | | Autism | | | |
| 5599 | Mapk8 | AMY-I | | | 10q11.22 | D MDD | | [symbol] |
| | mitogen activated protein | | | | BP | | | |
| | kinase 8 | | | | Panic Disorder^{]} | | | |
| | | | | | SZ | | | |
| 8938 | Baiap3 | AMY-D | | | 16p13.3 | D BP | | [symbol] |
| | BAI 1-associated protein 3 | | | | BP | | | |
| | | | | | Alcohol | | | |
| 1124 | Chn2 | AMY-D | | | 7p15.1 | D Alcohol | | [symbol] |
| | chimerin (chimaerin) 2 | | | | Neuroticism^{[} | | | |
| 26047 | Cntnap2 | AMY-D | | | 7q35 | D Alcoho | | [symbol] |
| | contactin associated | | | | Unipolar | | | |
| | protein-like 2 | | | | | | | |
| 29970 | Schip1 | AMY-I | | | 3q25.32 | D Alcohol | | [symbol] |
| | Schwannomin interacting | | | | BP | | | |
| | protein 1 | | | | Simple Phobia^{[} | | | |
| | | | | | SZA | | | |
| 4155 | Mbp | PFC-D | | | 18q23 | D BP | | [symbol] |
| | myelin basic protein | | | | BP | D SZ | | |
| | | | | | SZ | D Alcoho | | |
| | | | | | | I Alcohol^{[} | | |
| | | | | | | D Alzheimer | | |
| 6199 | Rps6kb2 | AMY-I | | | 11q13.2 | Alcohol^{[} | | [symbol] |
| | ribosomal protein S6 | | | | BP | | | |
| | kinase, polypeptide 2 | | | | SZ^{[} | | | |
| 6446 | Sgk | PFC-D | D | | 6q23.2 | | | [symbol] |
| | serum/glucocorticoid | | | | BP | | | |
| | regulated kinase | | | | SZ | | | |
| 54407 | Slc38s2 | PFC-D | D | | 12q13.11 | | | [symbol] |
| | solute carrier family 38, | | | | Neuroticism | | | |
| | member 2 | | | | Panic Disorder | | | |
| 2824 | Gpm6b | AMY-I | | | Xp22.2 | D Alcohol | D SZ | [symbol] |
| | Glycoprotein M6B | | | | | I Alcohol | | |
| | | | | | | I SZ | | |
| 794 | Calb2 | AMY-D | | | 16q22.2 | D SZ | | [symbol] |
| | calbindin 2 | | | | Alcohol | I SZ | | |
| 25864 | Abhd14a | AMY-D | D | | 3p21.2 | | | [symbol] |
| | abhydrolase domain | | | | | | | |
| | containing 14A | | | | | | | |
| 8905 | Ap1s2 | AMY-D | D | | Xp22.2 | | | [symbol] |
| | adaptor-related protein | | | | | | | |
| | complex 1, sigma 2 subunit | | | | | | | |
| | B230337E12Rik | AMY-I | D | | | | | |
| | RIKEN cDNA B230337E12 | | | | | | | |
| | gene | | | | | | | |
| 114569 | Mal2 | AMY-D | | | 8q23 | | | [symbol] |
| | mal, T-cell differentiation | PFC-D | | | BP | | | |
| | protein 2 | | | | SZA | | | |
| | | | | | Autism | | | |
| 91851 | Chrdl1 | AMY-I | | | Xq23 | I SZ | | [symbol] |
| | chordin-like 1 | | | | | | | |
| 3181 | Hnrpa2b1 | AMY-I | | | 7p15.2 | D SZ^{]} | | [symbol] |
| | heterogeneous nuclear | | | | | | | |
| | ribonucleoprotein A2/B1 | | | | | | | |
| 199731 | Igsf4c /// Cadm4 | AMY-I | | | 1q23.2 | D SZ | | [symbol] |
| | immunoglobulin | | | | | | | |
| | superfamily, member 4B | | | | | | | |

Italics—candidate blood biomarker genes.
I—Increased in expression;
D—decreased in expression.
BP—bipolar,
SZ—schizophrenia,
SZA—schizoaffective,
MDD—major depressive disorder,
PTSD—post traumatic stress disorder [symbol]
indicates data missing or illegible when filed

TABLE 5
Top DBP KO ST genes.

| | | Mouse | | | | | | |
|---|---|---|---|---|---|---|---|---|
| | | Brain region(s)- | | | | | | |
| | | Direction | | Mouse | | | | |
| | Gene Symbol- | of | | Quantitative Trait | Human Genetic | Human | Human | |
| Entrez ID | Description | change | Mouse Blood | Loci (QTL) | Evidence | Postmortem Brain | Blood | CFG Score |
| 6622 | Snca | AMY-D | D | Chr 6 | 4q22.1 | D Alcohol | I SZ | 5.5 |
| | synuclein, alpha | | | Abnormal fear/anxiety- | BP | I Alcohol | | |
| | | | | related behavior, | Alcohol [symbol] | | | |
| | | | | Depression-related | SZ | | | |
| | | | | behavior | Autism | | | |
| 6258 | Rxrg | PFC-D | | Chr 1 | 1q23.3 | D Alcohol^{[Lewohl et al.,} | I PTSD^{[Segman} | 5 |
| | retinoid X receptor gamma | | | Abnormal fear/anxiety- | BP^{[Fallin et al., 2004]} | ^{2000]} | ^{et al., 2005]} | |
| | | | | related behavior, | Autism^{[Ylisaukko-oja et al., 2006]} | | | |
| | | | | Addiction/drug abuse, | Alcohol^{[Hill et al., 2004], [Guerrini} | | | |
| | | | | Depression-related | ^{et al., 2005], [Kuo et al., 2006b]} | | | |
| | | | | behavior | SZ^{[Gurling et al., 2001]} | | | |
| 1813 | Drd2 | PFC-D | | Chr 9 | 11q23.92 | I BP^{[Ryan et al., 2006]} | I SZ^{[Zvara et al.,} | 4.5 |
| | dopamine receptor 2 | | | Abnormal fear/anxiety- | Alcohol^{[Sun et al., 1999]} | D Depression^{[Torrey et al.,} | ^{2005]} | |
| | | | | related behavior, | | ^{2005]} | | |
| | | | | Abnormal sleep | | D Alcohol^{[Noble et al., 1991]} | | |
| | | | | pattern/circadian rhythm | | D SZ^{[Seeman et al., 1997]}, | | |
| | | | | | | ^{[Dean et al., 2004], [Torrey et al.,} | | |
| | | | | | | ^{2005]} | | |
| | | | | | | I Tourette | | |
| | | | | | | Syndrome^{[Minzer et al.,} | | |
| | | | | | | ^{2004]} | | |
| | | | | | | D Marijuana^{[Wang et al.,} | | |
| | | | | | | ^{2004b]} | | |
| 3303 | Hspa1a | PFC-I | | Chr 17 | 6p21.3 | I SZ^{[Clark et al., 2006]} | I Stress^{[Ohmori} | 4.5 |
| | heat shock protein 1A | | | Anxiety/Fear | BP^{[Turecki et al., 2001]} | D Autism^{[Purcell et al., 2001]} | ^{et al., 2005]} | |
| | | | | | Psychosis^{[Kohn et al., 2004]} | | | |
| | | | | | Alcohol^{[Wyszynski et al., 2003]} | | | |
| | | | | | SZ^{[Lindholm et al., 2001], [Straub et} | | | |
| | | | | | ^{al., 2002a], [Fallin et al., 2003], [Suarez} | | | |
| | | | | | ^{et al., 2006],} | | | |
| 6421 | Sfpg | AMY-I | | Chr 4 | 1p34.3 | I BP^{[Nakatani et al., 2006]} | D SZ^{[Glatt et al.,} | 4.5 |
| | splicing factor | | | Addiction/drug abuse, | SZ^{[Straub et al., 2002b]} | | ^{2005]} | |
| | proline/glutamine rich | | | Abnormal fear/anxiety- | Anorexia Nervosa^{[Grice et al.,} | | | |
| | (polypyrimidine tract binding | | | related behavior, | ^{2002]} | | | |
| | protein associated) | | | Depression-related | | | | |
| | | | | behavior | | | | |
| 377 | Arf3 | PFC-D | | | 12q13.12 | D Alcohol^{[Lewohl et al.,} | D, Chronic | 4 |
| | ADP-ribosylation factor 3 | | | | Panic Disorder^{[Smoller et al.,} | ^{2000]} | Stress (^{Miller et} | |
| | | | | | ^{2001], [Fyer et al., 2006]} | | ^{al., 2008)} | |
| 2353 | Fos | AMY-I | | Chr 12 | 14q24.3 | | I PTSD^{[Segman} | 4 |
| | FBJ osteosarcoma | | | Abnormal sleep | SZ^{[Takahashi et al., 2005]} | | ^{et al., 2005]} | |
| | oncogene | | | pattern/circadian rhythm, | Alcohol^{[Hill et al., 2004]} | | | |
| | | | | Abnormal fear/anxiety- | Simple Phobia^{[Gelemter et al.,} | | | |
| | | | | related behavior | ^{2003]} | | | |
| 3716 | Jak1 | AMY-D | | Chr 4 | 1p31.3 | | I BP^{[Middleton et} | 4 |
| | Janus kinase 1 | | | Anxiety/Fear | BP^{[Rice et al., 1997], [Cichon et al.,} | | ^{al., 2005]} | |
| | | | | | ^{2001]} | | | |
| | | | | | Alcohol^{[Numberger et al., 2001],} | | | |
| | | | | | ^{[Schuckit et al., 2001],} | | | |
| | | | | | Depression^{[Numberger et al.,} | | | |
| | | | | | ^{2001]} | | | |
| 4118 | Mal | PFC-D | | | 2q11.1 | D Depression^{[Aston et al.,} | D BP^{[Middleton et} | 4 |
| | myelin and lymphocyte | | | | Alcohol^{[Wyszynski et al., 2003],} | ^{2005]} | ^{al., 2005]} | |
| | protein, T-cell differentiation | | | | ^{[Foroud et al., 2000], [Reich et al.,} | D Alcohol^{[Lewohl et al.,} | I BP^{[Matigian et al.,} | |
| | protein | | | | ^{1998]} | ^{2000]} | ^{2007]} | |
| | | | | | SZ^{[Lewis et al., 2003], [Straub et al.,} | D SZ^{[Davis et al., 2003],} | | |
| | | | | | ^{2002b], [DeLisi et al., 2002], [Chen et} | ^{[Mcinnes and Lauriat, 2006],} | | |
| | | | | | ^{al., 1998]} | ^{[Hakak et al., 2001]} | | |
| 5878 | Rab5c | AMY-I | | Chr 11 | 17q21.2 | Increase BP^{[Nakatani et al.,} | | 4 |
| | RAB5C, member RAS | | | Anxiety/Fear | Alcohol^{[Dick et al., 2006]} | ^{2006]} | | |
| | oncogene family | | | | Autism^{[Cantor et al., 2005]} | | | |
| 2554 | Gabra1 | PFC-D | | Chr 11 | 5q34-q35 | I SZ^{[Deng and Huang, 2006],} | | 4 |
| | gamma-aminobutyric acid | | | Abnormal fear/anxiety- | Alcohol^{[Dick et al., 2002a]} | [^{Hakak et al., 2001]} | | |
| | (GABA-A) receptor, subunit | | | related behavior, | BP^{[Morissette et al., 1999], [Sklar et} | D Multiple | | |
| | alpha 1 | | | Depression-related | ^{al., 2004]} | Sclerosis^{[Dutta et al., 2006]} | | |
| | | | | behavior, Addiction/drug | SZ^{[Sklar et al., 2004]} | I BP^{[Ishikawa et al., 2004]} | | |
| | | | | abuse | Psychosis^{[Sklar et al., 2004]} | D Suicide^{[Sequeira et al.,} | | |
| | | | | | | ^{2007]} | | |
| 5999 | Rgs4 | PFC-D | | Chr 1 | 1q23.3 | I Alcohol^{[Lewohl et al., 2000]} | | 4 |
| | regulator of G-protein | | | Abnormal fear/anxiety- | BP^{[Fallin et al., 2004], [Fallin et al.,} | D SZ^{[Prasad et al., 2005],} | | |
| | signalling 4 | | | related behavior, | ^{2005]} | ^{[Erdely et al., 2006], [Chowdari et} | | |
| | | | | Addiction/drug abuse, | Alcohol^{[Hill et al., 2004], [Guerrini} | ^{al., 2002], [Lipska et al., 2006],} | | |
| | | | | Depression-related | ^{et al., 2005], [Kuo et al., 2006b]} | ^{[Arion et al., 2007]} | | |
| | | | | behavior, | SZ^{[Brzustowicz et al., 2000], [Gurling} | D Alzheimers^{[Emilsson et} | | |
| | | | | | ^{et al., 2001], [Fallin et al., 2005]} | ^{al., 2006]} | | |
| | | | | | Autism^{[Auranen et al., 2002],} | | | |
| | | | | | ^{[Vorstman et al., 2006], [Ylisaukko-oja} | | | |
| | | | | | ^{et al., 2006]} | | | |
| 1428 | Crym | AMY-D | | Chr 7 | 16p12.2 | D Alcohol^{[Mayfield et al.,} | | 4 |
| | crystallin, mu | | | Abnormal fear/anxiety- | BP^{[Dick et al., 2002a], [Maziade et} | ^{2002],} | | |
| | | | | related behavior, | ^{al., 2005], [Cheng et al., 2006]} | D SZ^{[Arion et al., 2007]} | | |
| | | | | Abnormal sleep | SZ^{[Maziade et al., 2005]} | I SZ^{[Hakak et al., 2001]} | | |
| | | | | pattern/circadian rhythm | Panic Disorder^{[Crowe et al.,} | D Alzheimers^{Emilsson et} | | |
| | | | | | ^{2001]} | al., 2006] | | |
| 2670 | Gfap | AMY-I | | Chr 11 | 17q21.31 | D BP^{[Tkachev et al., 2003],} | | 4 |
| | glial fibrillary acidic protein | | | Anxiety/Fear | Alcohol^{[Dick et al., 2006]} | ^{[Webster et al., 2005]} | | |
| | | | | | SZ^{[Lewis et al., 2003]} | D Depression^{[Fatemi et al.,} | | |
| | | | | | SZA^{[Vincent et al., 1999]} | ^{2004]} | | |
| | | | | | Autism^{[Cantor et al., 2005]} | D Alcohol^{[Lewohl et al.,} | | |
| | | | | | | ^{2000], [Liu et al., 2004], [Mayfield} | | |
| | | | | | | ^{et al., 2002]} | | |
| | | | | | | I SZ^{[Tkachev et al., 2003]} | | |
| | | | | | | D SZ^{[Clark et al., 2006],} | | |
| | | | | | | ^{[Vawter et al., 2001], [Webster et} | | |
| | | | | | | ^{al., 2005]} | | |
| | | | | | | I Autism^{[Purcell et al., 2001]} | | |
| 5295 | Pik3r1 | PFC-D | | chr 13, 50.0 | 5q13.1 | D MDD^{[Aston et al., 2005]} | | 4 |
| | phosphatidylinositol 3- | | | Addiction/drug abuse, | Psychosis^{[Kohn et al., 2004]} | | | |
| | kinase, regulatory subunit, | | | Abnormal fear/anxiety- | Alcohol^{[Hill et al., 2004]} | | | |
| | polypeptide 1 (p85 alpha) | | | related behavior, | SZ^{[Suarez et al., 2006]} | | | |
| | | | | Depression-related | | | | |
| | | | | behavior | | | | |
| I1122 | Ptprt | AMY-I | | Chr 2 | 20q12 | D MDD^{[Aston et al., 2005]} | | 4 |
| | Protein tyrosine | | | Anxiety/Fear | BP^{[Radhakrishna et al., 2001]} | | | |
| | phosphatase, receptor type, T | | | | Alcohol^{[Hill et al., 2004]} | | | |
| 6857 | Syt1 | PFC-D | | Chr 10 | 12q21.2 | D BP^{[Ryan et al., 2006]} | | 4 |
| | synaptotagmin I | | | Abnormal fear/anxiety- | BP^{[Morissette et al., 1999]} | D Alcohol^{[Flatscher-Bader et} | | |
| | | | | related behavior, | | ^{al., 2005]} | | |
| | | | | Abnormal sleep | | D Heroin^{[Albertson et al.,} | | |
| | | | | pattern/circadian rhythm | | ^{2006]} | | |
| | | | | | | D SZ^{[Hemby et al., 2002]} | | |
| | | | | | | D SZ^{[Sokolov et al., 2000]} | | |
| 2571 | Gad1 | PFC-D | | Chr 2 | 2q31.1 | D Epilepsy^{[Arion et al., 2006]} | | 4 |
| | glutamic acid decarboxylase 1 | | | Abnormal sleep | BP^{[Cichon et al., 2001], [Cheng et al.,} | D BP^{[Konradi et al., 2004]} | | |
| | | | | pattern/circadian rhythm, | ^{2006]} | | | |
| | | | | Depression-related | Alcohol^{[Schuckit et al., 2001]} | | | |
| | | | | behavior, Abnormal | | | | |
| | | | | fear/anxiety-related | | | | |
| | | | | behavior | | | | |
| 1267 | Cnp | PFC-D | | Chr 11 | 17q21.2 | D Depression^{[Aston et al.,} | | 4 |
| | cyclic nucleotide | | | Anxiety/Fear | BP^{[Segurado et al., 2003]} | ^{2005]} | | |
| | phosphodiesterase 1 | | | | Alcohol^{[Dick et al., 2006]} | D Alcohol^{[Lewohl et al.,} | | |
| | | | | | SZ^{[Lewis et al., 2003], [Peirce et al.,} | ^{2000], [Liu et al., 2006]} | | |
| | | | | | ^{2006]} | D SZ^{[Davis et al.,} | | |
| | | | | | Austism^{[Cantor et al., 2005]} | ^{2003],[Dracheva et al.,} | | |
| | | | | | | ^{2005],[Flynn et al., 2003],[Hakak} | | |
| | | | | | | ^{et al., 2001],[McCullumsmith et} | | |
| | | | | | | ^{al., 2007],[McInness and Lauriat,} | | |
| | | | | | | ^{2006],[Peirce et al., 2006], [Aston} | | |
| | | | | | | ^{et al., 2004]} | | |
| 1822 | Atn1 | PFC-I | | Chr 6 | 12p13.31 | D BP^{[Nakatani et al., 2006]} | | 4 |
| | atrophin 1 | | | Abnormal fear/anxiety | Alcohol^{[Hill et al., 2004]} | | | |
| | | | | related behavior, | | | | |
| | | | | Depression-related | | | | |
| | | | | behavior, Addiction/drug | | | | |
| | | | | abuse | | | | |
| 1159 | Ckmt1 | AMY-D | | Chr 2 | 15q15.3 | D BP^{[Jurata et al., 2004]} | | 4 |
| | creatine kinase | | | Anxiety/Fear | Alcohol^{[Dick et al., 2002b]} | | | |
| | mitochondrial 1 (ubiquitous | | | | SZ^{[Freedman et al., 2001], [Maziade} | | | |
| | | | | | ^{et al., 2005], [Stober et al., 2000]} | | | |
| 2770 | Gnai1 | PFC-D | | Chr 5 | 7q21.11 | D BP^{[Jurata et al., 2004]} | | 4 |
| | guanine nucleotide binding | | | Abnormal fear/anxiety- | BP^{[Lambert et al., 2005]} | | | |
| | protein, alpha inhibiting 1 | | | related behavior | Alcohol^{[Wang et al., 2005]} | | | |
| | | | | | Panic Disorder^{[Cheng et al.,} | | | |
| | | | | | ^{2006]} | | | |
| | | | | | Autism^{[Barrett et al., 1999],} | | | |
| | | | | | ^{[Vorstman et al., 2006], [Liu et al.,} | | | |
| | | | | | ^{2001]} | | | |
| 793 | Calb1 | AMY-D | | Chr 4 | 8q21.3 | D BP, SZ^{[Torrey et al., 2005]} | | 4 |
| | calbindin-28K | | | Addiction/drug abuse | BP^{[Liu et al., 2003]} | BP^{[Shamir et al., 2005]} | | |
| | | | | | | I SZ^{[Weidenhofer et al., 2006],} | | |
| | | | | | | ^{[Iritani et al., 1999]} | | |
| | | | | | | Alzheimer^{[Ferrer et al.,} | | |
| | | | | | | ^{1993]} | | |
| 4340 | Mog | PFC-D | | Chr 17 | 6p22.1 | D BP^{[Tkachev et al., 2003]} | | 4 |
| | myelin oligodendrocyte | | | Anxiety/Fear | BP^{[Schulze et al., 2004], [Turecki et} | D Depression^{[Aston et al.,} | | |
| | glycoprotein | | | | ^{al., 2001]} | ^{2005]} | | |
| | | | | | Alcohol^{[Wyszynski et al., 2003]} | D Alcohol^{[Lewohl et al.,} | | |
| | | | | | Psychosis^{[Kohn et al., 2004]} | ^{2000]} | | |
| | | | | | SZ^{[Straub et al., 2002b], [Suarez et} | D SZ^{[Tkachev et al., 2003],} | | |
| | | | | | al., 2006] | ^{[McInness and Lauriat, 2006]} | | |
| | | | | | | I Epilepsy^{[Arion et al., 2006]} | | |
| 7881 | Kcnab1 | PFC-D | | Chr 3 | 3q25.31 | D Alcohol^{[Sokolov et al.,} | | 4 |
| | potassium voltage-gated | | | Abnormal fear/anxiety- | BP^{[Badenhop et al., 2002], [Curtis et} | ^{2003a]} | | |
| | channel, shaker-related | | | related behavior | ^{al., 2003]} | | | |
| | subfamily, beta member 1 | | | | SZA^{[Barndenhop et al., 2002]} | | | |
| | | | | | Simple Phobia^{[Gelemter et al.,} | | | |
| | | | | | ^{2003]} | | | |
| | | | | | Agoraphobia^{[Gelernter et al.,} | | | |
| | | | | | ^{2001]} | | | |
| 8405 | Spop | PFC-D | | Chr 11 | 17q21.33 | D Alcohol^{[Sokolov et al.,} | | 4 |
| | speckle-type POZ protein | | | Abnormal fear/anxiety- | Alcohol^{[Dick et al., 2006]} | ^{2003a]} | | |
| | | | | related behavior, | Autism^{[Cantor et al., 2005]} | | | |
| | | | | Abnormal sleep | | | | |
| | | | | pattern/circadian rhythm, | | | | |
| | | | | Addiction/drug abuse | | | | |
| 5796 | Ptprk | AMY-D | | Chr 10 | 6q22.33 | D Alcohol^{[Lewohl et al.,} | | 4 |
| | protein tyrosine | | | Anxiety/Fear (QTL) | BP^{[Park et al., 2004]} | ^{2000]} | | |
| | phosphatase, receptor type, K | | | | Alcohol^{[Sun et al., 1999]} | | | |
| | | | | | SZ^{[Straub et al., 2002b]} | | | |
| 1282 | Col4a1 | PFC-I | | Chr 8 | 13q34 | D Alcohol^{[Flatscher-Bader et} | | 4 |
| | procollagen, type IV, alpha 1 | | | Abnormal fear/anxiety- | BP^{[Maziade et al., 2005], [Kelsoe et} | ^{al., 2005]} | | |
| | | | | related behavior, | ^{al., 2001]} | | | |
| | | | | Addiction/drug abuse, | | | | |
| | | | | Depression-related | | | | |
| | | | | behavior | | | | |
| 7077 | Timp2 | PFC-D | | Chr 11 | 17q25 | D Alcohol^{[Flatscher-Bader et} | | 4 |
| | tissue inhibitor of | | | Abnormal fear/anxiety- | MDD^{[Curtis et al., 2003]} | ^{al., 2005]} | | |
| | metalloproteinase 2 | | | related behavior, | | D Alcohol^{[Liu et al., 2006]} | | |
| | | | | Addiction/drug abuse | | | | |
| 347 | Apod | AMY-I | | Chr 16 | 3q29 | D MDD^{(Aston et al. 2005)} | | 3.5 |
| | apolipoprotein D | PFC-D | | Abnormal Emotion/Affect | Alzheimer Disease | I BP^{(Thomas et al. 2003)} | | |
| | | | | Behavior | | D Alcohol^{(Lewohl et al.} | | |
| | | | | | | ²⁰⁰⁰⁾ | | |
| | | | | | | I SZ^{(Thomas et al. 2003)} | | |
| 1958 | Egr1 | AMY-I | | Chr 18 | 5q31.2 | D SZ^{[Yamada et al., 2007]} | I SZ^{[Middleton et} | 3.5 |
| | early growth response 1 | | | Addiction/drug abuse, | SZ^{[Straub et al., 1997],[Devlin et al.,} | | ^{al., 2005]} | |
| | | | | Depression-related | ^{2002]} | | | |
| | | | | behavior, Abnormal | | | | |
| | | | | fear/anxiety-related | | | | |
| | | | | behavior, Abnormal | | | | |
| | | | | sleep pattern/circadian | | | | |
| | | | | rhythm | | | | |
| 2782 | Gnb1 | PFC-I | | Chr 4 | 1p36.33 | I SZ^{[Clark et al., 2006]} | I BP^{[Middleton et} | 3.5 |
| | guanine nucleotide binding | | | Abnormal fear/anxiety- | | D SZ^{[Hemby et al., 2002]} | ^{al., 2005]} | |
| | protein, beta 1 | | | related behavior | | | | |
| 4023 | Lpl | AMY-D | | Chr 8 | 8p21.3 | I SZ^{[Glatt et al., 2005]} | | 3.5 |
| | lipoprotein lipase | PFC-D | | Abnormal sleep | SZ^{[Maziade et al., 2005], [Kendler et} | | | |
| | | | | pattern/circadian rhythm, | ^{al., 1996], [Straub et al., 2002b],} | | | |
| | | | | Addiction/drug abuse, | ^{[Suarez et al., 2006], [Cheng et al.,} | | | |
| | | | | Depression-related | ^{2006], [Brzustowicz et al., 2000],} | | | |
| | | | | behavior | ^{[Brzustowicz et al., 1999], [Gurling et} | | | |
| | | | | | ^{al., 2001], [Blouin et al., 1998], [Pulver} | | | |
| | | | | | ^{et al., 2000], [Chiu et al., 2002]} | | | |
| 7008 | Tef | PFC-D | | Chr 15 | 22q13.2 | | | 3.0 |
| | thyrotroph embryonic factor | | | Abnormal Emotion/Affect | BP^{(Kelsoe et al. 2001) (Baron et al.} | | | |
| | | | | Behavior | ²⁰⁰¹⁾ | | | |
| | | | | Abnormal Circadian | Panic Disorder^{(Hamilton et al.} | | | |
| | | | | Rhythm | ²⁰⁰³⁾ | | | |
| | | | | Addiction/Drug Abuse | | | | |
| 1465 | Csrp1 | PFC-D | | | 1q32.1 | D Alcohol^{[Lewohl et al.,} | | 3 |
| | cysteine and glycine-rich | | | | Alcohol^{[Sun et al., 1999]} | ^{2000], [Sokolov et al., 2003a]} | | |
| | protein 1 | | | | Panic Disorder^{[Smoller et al.,} | I Epilepsy^{[Arion et al., 2006]} | | |
| | | | | | ^{2001]} | D SZ^{[Hakak et al., 2001]} | | |
| | | | | | Anorexia Nervosa^{[Devlin et} | | | |
| | | | | | ^{al., 2002]} | | | |
| | | | | | SZ^{[Paunio et al., 2004]} | | | |
| I1342 | Rnf13 | PFC-D | | | 3q25.1 | I BP^{[Nakatani et al., 2006]} | | 3 |
| | ring finger protein 13 | | | | BP^{[Badenhop et al., 2002], [Curtis et} | | | |
| | | | | | ^{al., 2003]} | | | |
| | | | | | SZA^{[Badenhop et al., 2002]} | | | |
| | | | | | Simple Phobia^{[Gelemter et al.,} | | | |
| | | | | | ^{2003]} | | | |
| | | | | | Agoraphobia^{[Gelernter et al.,} | | | |
| | | | | | ^{2001]} | | | |
| 7184 | Tra1 | PFC-D | | | 12q23.3 | I BP^{[Jurata et al., 2004]} | | 3 |
| | tumor rejection antigen | | | | BP^{[Maziade et al., 2005]} | | | |
| | gp96 | | | | Alcohol^{[Hill et al., 2004]} | | | |
| | | | | | SZ^{[Maziade et al., 2005]} | | | |
| 8899 | Prpf4b | PFC-I | | | 6p25.2 | I BP^{[Iwamoto et al., 2004]} | | 3 |
| | PRP4 pre-mRNA | | | | Alcohol^{[Hill et al., 2004]} | | | |
| | processing factor 4 homolog | | | | SZ^{[Straub et al., 1995], [Maziade et} | | | |
| | B (yeast) | | | | ^{al., 1997]} | | | |
| 5567 | Prkacb | PFC-D | | | 1p31.1 | D Alcohol^{[Lewohl et al.,} | | 3 |
| | protein kinase, cAMP | | | | Depression^{[Nurnberger et al.,} | ^{2000]}I Alzheimers^{[Emisson} | | |
| | dependent, catalytic, beta | | | | ^{2001]} | ^{et al., 2006]} | | |
| | | | | | BP^{[Rice et al., 1997]} | | | |
| | | | | | Alcohol^{[Reich et al., 1998],} | | | |
| | | | | | ^{[Peterson et al., 1999],[Foroud et al.,} | | | |
| | | | | | ^{2000], [Numberger et al., 2001],} | | | |
| | | | | | ^{[Schuckit et al., 2001], [Guerrini et al.,} | | | |
| | | | | | ^{2005]} | | | |
| | | | | | SZ^{[Brzustowicz et al., 2000]} | | | |
| 2932 | Gsk3b | AMY-I | | | 3q13.33 | D SZ^{[Kozlovsky et al., 2000],} | | 3 |
| | glycogen synthase kinase 3 | PFC-D | | | BP^{[Maziade et al., 2005]} | ^{[Torrey et al., 2005]} | | |
| | beta | | | | | D BP^{[Nakatani et al., 2006],} | | |
| | | | | | | ^{[Vawter et al., 2006]} | | |
| | | | | | | I MDD^{[Vawter et al., 2006]} | | |
| 8073 | Ptp4a2 | PFC-D | | | 1p35.2 | D MDD^{[Aston et al., 2005]} | | 3 |
| | protein tyrosine | | | | BP^{[Cichon et al., 2001]} | I SZ^{[Vawter et al., 2004]} | | |
| | phosphatase 4a2 | | | | SZ^{[Straub et al., 2002b]} | I Suicide^{[Sequeira et al.,} | | |
| | | | | | | ^{2007]} | | |
| I0094 | Arpc3 | PFC-I | | | 12q24.11 | D BP^{[Konradi et al., 2004]} | | 3 |
| | actin related protein 2/3 | | | | BP^{[Chagnon et al., 2004]} | | | |
| | complex, subunit 3 | | | | Alcohol^{[Hill et al., 2004]} | | | |
| | | | | | SZ^{[Fallin et al., 2003]} | | | |
| I0059 | Dnm1l | PFC-D | | | 12p11.21 | D BP^{[Konradi et al., 2004]} | | 3 |
| | dynamin 1-like | | | | Neuroticism^{[Neale et al., 2005]} | | | |
| | | | | | SZ^{[Takahashi et al., 2005]} | | | |
| | | | | | Panic Disorder^{[Fyer et al.,} | | | |
| | | | | | ^{2006], [Smoller et al., 2001]} | | | |
| 9867 | Pja2 | PFC-D | | | 5q21.3 | D BP^{[Ryan et al., 2006]} | | 3 |
| | praja 2, RING-H2 motif | | | | Alcohol^{[Hill et al., 2004]} | | | |
| | containing | | | | | | | |
| 5010 | Cldn11 | PFC-D | | | 3q26.2 | D BP^{[Tkachev et al., 2003]} | | 3 |
| | claudin 11 (oligodendrocyte | | | | BP^{[Cichon et al., 2001]} | D SZ^{[Tkachev et al., 2003],} | | |
| | transmembrane protein) | | | | SZ^{[DeLisi et al., 2002]} | ^{[McInnes and Lauriat, 2006],} | | |
| | | | | | | ^{[Dracheva et al., 2005]} | | |
| | | | | | | I SZ^{[Weidenhofer et al., 2006]} | | |
| 2913 | Grm3 | AMY-I | | | 7q21.12 | D BP^{[Choudary et al., 2005]} | | 3 |
| | Glutamate receptor, | | | | BP^{[Lambert et al., 2005]} | SZ^{[Hemby et al., 2002]} | | |
| | metabotrophic 3 | | | | Alcohol^{[Wang et al., 2005], [Foroud} | | | |
| | | | | | ^{et al., 2000]} | | | |
| | | | | | Panic Disorder^{[Cheng et al.,} | | | |
| | | | | | ^{2006]} | | | |
| | | | | | Autism^{[Barrett et al., 1999],} | | | |
| | | | | | ^{[Vorstman et al., 2006], [Liu et al.,} | | | |
| | | | | | ^{2001]} | | | |
| 2941 | Gsta4 | AMY-D | | | 6p12.1 | D BP^{[Benes et al., 2005]} | | 3 |
| | glutathione S-transferase, | | | | BP^{[Lambert et al., 2005]} | I BP^{[Nakatani et al., 2006]} | | |
| | alpha 4 | | | | | | | |
| 4336 | Mobp | AMY-I | | Chr 9 | 3p22.2 | D BP, SZ^{[Tkachev et al.,} | | 3 |
| | myelin-associated | PFC-D | | Abnormal Sleep | | ^{2003]} | | |
| | oligodendrocyte basic | | | pattern/circadian rhythm | | D MDD^{[Aston et al., 2005]} | | |
| | protein | | | | | D Alcohol^{[Lewohl et al.,} | | |
| | | | | | | ^{2000]} | | |
| | | | | | | I Alcohol^{[Mayfield et al.,} | | |
| | | | | | | ^{2002]} | | |
| 4753 | Nell2 | PFC-I | | | 12q12 | I Alcohol^{[Lewohl et al., 2000]} | | 3 |
| | nel-like 2 homolog (chicken) | | | | Neuroticism^{[Neale et al., 2005]} | D Alzheimers^{[Emilsson et} | | |
| | | | | | SZ^{[Takahashi et al., 2005]} | ^{al., 2006]} | | |
| | | | | | Panic Disorder^{[Fyer et al.,} | I SZ^{[Hakak et al., 2001]} | | |
| | | | | | ^{2006], [Smoller et al., 2001]} | | | |
| [symbol] 3040 | Myt1l | PFC-D | | | 2p25.3 | D Alcohol^{[Liu et al., 2004]} | | 3 |
| | myelin transcription factor 1- | | | | BP^{[Detera-Wadleigh et al., 1999]} | | | |
| | like | | | | Panic Disorder^{[Hamilton et al.,} | | | |
| | | | | | ^{2003]} | | | |
| | | | | | SZ^{[Cardno et al., 2001]} | | | |
| 7086 | Tkt | PFC-D | | | 3p21.1 | D Alcohol^{[Liu et al., 2006]} | | 3 |
| | transketolase | | | | Alcohol^{[Foroud et al., 2000]} | | | |
| | | | | | SZ^{[Macgregor et al., 2004]} | | | |
| 4155 | Mbp | PFC-D | | | 18q23 | D BP^{[Tkachev et al., 2003]} | | 3 |
| | myelin basic protein | AMY-I | | | BP^{[Coon et al., 1996], [Freimer et al.,} | D Alcohol^{[Lewohl et al.,} | | |
| | | | | | ^{1996], [Ewald et al., 1999], [Schulze et} | ^{2000]} | | |
| | | | | | ^{al., 2003], [Maziade et al., 2005]} | I Alcohol^{[Liu et al., 2004]} | | |
| | | | | | SZ^{[Straub et al., 2002b],[Lewis et al.,} | D SZ^{[Tkachev et al., 2003]} | | |
| | | | | | ^{2003]} | D Alzheimer^{[Wang et al.,} | | |
| | | | | | | ^{2004a]} | | |
| 1124 | Chn2 | PFC-D | | | 7p15.1 | D Alcohol^{[Flatscher-Bader et} | | 3 |
| | chimerin (chimaerin) 2 | | | | Neuroticism^{[Nash et al., 2004]} | ^{al., 2005]} | | |
| 8531 | Csda | AMY-I | | | 12p13.2 | | I SZ^{[Glatt et al.,} | 2.5 |
| | cold shock domain protein A | | | | Alcohol^{[Hill et al., 2004]} | | ^{2005]} | |
| [symbol] 3608 | Mkrn1 | AMY-I | | | 7q34 | | I SZ^{[Glatt et al.,} | 2.5 |
| | makorin, ring finger protein, 1 | | | | Unipolar^{[Curtis et al., 2003]} | | ^{2005]} | |
| 6605 | Smarce1 | AMY-I | | | 17q21.2 | | D SZ^{[Middleton et} | 2.5 |
| | SWI/SNF related, matrix | | | | Alcohol^{[Dick et al., 2006]} | | ^{al., 2005]} | |
| | associated, actin dependent | | | | Autism^{[Cantor et al., 2005]} | | | |
| | regulator of chromatin, | | | | | | | |
| | subfamily e, member 1 | | | | | | | |
| 394 | Arhgap5 | PFC-D | | | 14q12 | I SZ^{[Glatt et al., 2005]} | | 2.5 |
| | Rho GTPase activating | | | | Alcohol^{[Hill et al., 2004]} | | | |
| | protein 5 | | | | SZ^{[Lerer et al., 2003]} | | | |
| I0645 | Camkk2 | PFC-D | | | 12q24.31 | D SZ^{[Glatt et al., 2005]} | | 2.5 |
| | calcium/calmodulin- | | | | SZ^{[Fallin et al., 2003]} | | | |
| | dependent protein kinase | | | | BP^{[Morissette et al., 1999],[Chagnon} | | | |
| | kinase 2, beta | | | | ^{et al., 2004]} | | | |
| | | | | | Unipolar^{[Curtis et al., 2003]} | | | |
| | | | | | Alcohol^{[Hill et al., 2004]} | | | |
| 794 | Calb2 | PFC-D | | | 16q22.2 | D SZ^{[Beasley et al., 2002]} | | 2.5 |
| | calbindin 2 | | | | Alcohol^{[Sheffield et al., 1999]} | I SZ^{[Weldenhofer et al., 2006]} | | |
| [symbol] 4112 | Gpr88 | PFC-D | | | 1p21.2 | | | 2.0 |
| | G-protein coupled receptor | | | | Alcohol^{(Schuckit et al. 2001)} | | | |
| | 88 | | | | MDD^{(Numberger et al. 2001)} | | | |
| | | | | | SZ^{(Brzustowicz et al. 2004)} | | | |
| [symbol] 1191 | Herc5 | PFC-I | | | 4q22.1 | | D, Chronic | 2.0 |
| | hect domain and RLD 5 | | | | | | Stress (^{Miller et} | |
| | | | | | | | ^{al. 2008)} | |
| 8204 | Nrip1 | PFC-D | | | 21q11.2 | | D PTSD^{[Segman} | 2 |
| | nuclear receptor interacting | | | | | | ^{et al., 2005]} | |
| | protein 1 | | | | | | | |
| [symbol] 6675 | Nrip3 | PFC-D | | | 11p15.3 | | D BP^{[Middleton et} | 2 |
| | nuclear receptor interacting | AMY-D | | | | | ^{al., 2005]} | |
| | protein 3 | | | | | | | |
| 5566 | Prkaca | PFC-I | | | 19p13.12 | D SZ^{[Glatt et al., 2005]} | | 2 |
| | protein kinase, cAMP | | | | SZA^{[Hamshere et al., 2005]} | | | |
| | dependent, catalytic, alpha | | | | | | | |
| 5354 | Plp1 | PFC-D | | | Xq22.2 | D BP^{[Tkachev et al., 2003]} | | 2 |
| | proteolipid protein (myelin) 1 | | | | | D Depression^{[Aston et al.,} | | |
| | | | | | | ^{2005]} | | |
| | | | | | | D Alcohol^{[Liu et al., 2006]} | | |
| | | | | | | D SZ^{[Aberg et al., 2006],} | | |
| | | | | | | ^{[Pongrac et al., 2002], Mclnnes} | | |
| | | | | | | ^{and Lauriat, 2006], [Tkachev et} | | |
| | | | | | | ^{al., 2003]} | | |
| 3181 | Hnrpa2b1 | PFC-I | | | 7p15.2 | | D SZ^{[Glatt et al.,} | 1.5 |
| | heterogeneous nuclear | | | | | | ^{2005]} | |
| | ribonucleoprotein A2/B1 | | | | | | | |
| 15207 | Kctd12 | AMY-D | | | 13q22.3 | | D SZ^{[Glatt et al.,} | 1.5 |
| | potassium channel | | | | | | ^{2005]} | |
| | tetramerisation domain | | | | | | | |
| | containing 12 | | | | | | | |
| 92683 | Scamp5 | PFC-D | | | 15q24.1 | D SZ^{[Glatt et al., 2005]} | | 1.5 |
| | secretory carrier membrane | | | | | | | |
| | protein 5 | | | | | | | |

Italics—candidate blood biomarker genes.
I—Increased in expression;
D—decreased in expression.
BP—bipolar,
SZ—schizophrenia,
SZA—schizoaffective,
MDD—major depressive disorder,
PTSD—post traumatic stress disorder.
The top blood biomarkers/candidate genes for which there was a reversal (normalization) of the direction of changes in expression on high DHA vs. low DHA diet are underlined. [symbol]
indicates data missing or illegible when filed

TABLE 6
DBP Mouse Brain-Blood Biomarkers

| | | | | | Mouse | | | | |
|---|---|---|---|---|---|---|---|---|---|
| | | NST | NST | NST | Quatitative | Human | Human | | |
| Entrez | Gene Symbol/ | DBP | DBP | DBP | Trait Loci | Genetic | Postmortem | Human | CFG |
| ID | Description | Blood | PFC | AMY | (QTL) | Evidence | Brain | blood | Score |
| 1267 | Cnp | D | D | | Chr 11 | Alcohol^{[Dick et al., 2006]} | D | | 5 |
| | cyclic nucleotide | | | | Abnormal | Autism^{[Cantor et al., 2005]} | Depression^{[Aston et al., 2005]} | | |
| | phosphodiesterase 1 | | | | fear/anxiety- | | D Alcohol^{[Lewohl et al., 2000; Liu et al., 2006]} | | |
| | | | | | related | | D | | |
| | | | | | behavior | | SZ^{[Davis et al., 2003; Dracheva et al., 2005; Flynn et al., 2003; Peirce et al., 2006]} | | |
| | | | | | Abnormal | | | | |
| | | | | | sleep | | | | |
| | | | | | pattern/ | | | | |
| | | | | | Circadian | | | | |
| | | | | | rhythm | | | | |
| | | | | | Addiction | | | | |
| 9987 | Hnrpdl | D | | I | Chr 5 | Alcohol^{[Reich et al., 1998]} | | | 4 |
| | heterogeneous nuclear | | | | Abnormal | SZ^{[Paunio et al., 2004]} | | | |
| | ribonucleoprotein | | | | sleep | | | | |
| | D-like | | | | pattern/ | | | | |
| | | | | | circadian | | | | |
| | | | | | rhythm | | | | |
| 7534 | Ywhaz | I | I | I | | | I BP^{[Nakatani et al., 2006]} | | 3 |
| | tyrosine 3- | | | | | | D Alcohol^{[Flastcher-Bader et al., 2005]} | | |
| | monooxygenase/ | | | | | | D SZ^{[Glatt et al., 2005]} | | |
| | tryptophan | | | | | | | | |
| | 5-monooxygenase | | | | | | | | |
| | activation protein, zeta | | | | | | | | |
| | polypeptide | | | | | | | | |
| 6446 | Sgk | D | D | | | BP^{[Ewald et al., 2002; Venken et al., 2005]} | | | 3 |
| | serum/glucocorticoid | | | | | SZ^{[Takahashi et al., 2005]} | | | |
| | regulated kinase | | | | | | | | |
| 54407 | Slc38a2 | D | D | | | Neuroticism^{[Neale et al., 2005]} | | | 3 |
| | solute carrier family | | | | | Panic | | | |
| | 38, member 2 | | | | | Disorder^{[Fyer et al., 2006]} | | | |
| 25864 | Abhd14a | D | | D | | | | | 2 |
| | abhydrolase domain | | | | | | | | |
| | containing 14A | | | | | | | | |
| 8905 | Ap1s2 | D | | D | | | | | 2 |
| | adaptor-related protein | | | | | | | | |
| | complex 1, sigma 2 | | | | | | | | |
| | subunit | | | | | | | | |
| | B230337E12Rik | D | | I | | | | | 2 |
| | RIKEN cDNA | | | | | | | | |
| | B230337E12 gene | | | | | | | | |
| | | | | | | Human | | | |
| | | ST | ST | ST | | Genetic | Human | | |
| | Gene Symbol/ | DBP | DBP | DBP | | Linkage | Brain | | |
| | Description | Blood | PFC | AMY | | Evidence | Evidence | | |
| 6622 | Snca | D | | D | Chr 6 | BP^{[Curtis et al., 2003]} | D | I SZ^{[Glatt et al., 2005]} | 5.5 |
| | synuclein, alpha | | | | Abnormal | Alcohol^{[Foroud et al., 2007; Reich et al., 1998; Williams et al., 1999]} | Alcohol^{[Lewohl et al., 2004; Mayfield et al., 2002]} | | |
| | | | | | fear/anxiety- | SZ^{[Paunio et al., 2004]} | | | |
| | | | | | related | Autism^{[Buxbaum et al., 2004]} | | | |
| | | | | | behavior | | | | |
| | | | | | Depression- | | | | |
| | | | | | related | | | | |
| | | | | | behavior | | | | |

I—Increased in expression;
D—decreased in expression.
Red—candidate blood biomarker genes.
BP—bipolar,
SZ—schizophrenia.
PFC—prefrontal cortex;
AMY—amygala.
The top blood biomarkers/candidate genes for which there was a reversal (normalization) of the direction of changes in expression on high DHA vs. low DHA diet are underlined.

TABLE 7
Broad/MIT Connectivity Map Results. Interogation with gene
expression pattern of genes switched from NST to ST

| | | | | |
|---|---|---|---|---|
| rank | cmap name | dose | cell line | score |

(a) PFC

| | | | | |
|---|---|---|---|---|
| 1 | celecoxib | 10 ÂμM | PC3 | 1 |
| 2 | monorden | 100 nM | PC3 | 0.998 |
| 452 | valproic acid | 1 mM | PC3 | −0.964 |
| 453 | 12,13-EODE | 200 nM | MCF7 | −1 |

(b) AMY

| | | | | |
|---|---|---|---|---|
| 1 | arachidonyltrifluoromethane | 10 ÂμM | MCF7 | 1 |
| 2 | 15-delta prostaglandin J2 | 10 ÂμM | MCF7 | 0.908 |
| 452 | 17-allylamino-geldanamycin | 1 ÂμM | SKMEL5 | −0.949 |
| 453 | iloprost | 1 ÂμM | MCF7 | −1 |

TABLE 8
Gene Ontology (GO) analysis. Genes changed in (a) DBP NST; (b)
DBP ST

| | Number of |
|---|---|
| | genes |
| a. GO Analysis-Biological Processes | |
| NST Data | |
| 1. Cellular Physiological Process | 315 |
| 2. Metabolism | 217 |
| 3. Cell Communication | 156 |
| 4. Regulation of Biological Process | 119 |
| 5. Localization | 116 |
| 6. Organismal Physiological Process | 81 |
| 7. Anatomical Structure Development | 79 |
| 8. Response to Stimulus | 70 |
| 9. Death | 30 |
| 10. Homeostasis | 17 |
| 11. Cell Adhesion | 13 |
| 12. Locomotion | 12 |
| 13. Cell Recognition | 7 |
| 13. Growth | 7 |
| 14. Sexual Reproduction | 5 |
| 14. Pattern Specification | 5 |
| 14. Rhythmic Process | 5 |
| 15. Embryonic Development | 4 |
| 15. Extracellular Structure organization and Biogenesis | 4 |
| 16. Reproductive Physiological Process | 3 |
| 16. Developmental Maturation | 3 |
| 17. Lysogeny | 2 |
| 17. Interaction Between Organisms | 2 |
| 18. Physiological Interaction Between Organisms | 1 |
| 18. Pigmentation During Development | 1 |
| 18. Post-embryonic Development | 1 |
| Response to Stress | |
| Response to Endogenous Stimulus | |
| Behavior | |
| Response to Chemical Stimulus | |
| Response to External Stimulus | |
| Defense Response | |
| Response to abiotic Stimulus | |
| Response to biotic Stimulus | |
| Coagulation | |
| Segmentation | |
| b. GO Analysis-Biological Processes | |
| ST Data (rank of biological process category in DBP | |
| NST analysis). | |
| 1. Cellular Physiological Process (1) | 464 |
| 2. Cell Communication (3) | 186 |
| 4. Anatomical Structure Development (7) | 127 |
| 5. Organismal Physiological Process (6) | 105 |
| 3. Metabolism (2) | 82 |
| 6. Regulation of Biological Process (4) | 53 |
| 7. Localization (5) | 42 |
| 8. Death (9) | 38 |
| 9. Response to Stress (unranked) | 33 |
| 10. Behavior (unranked) | 28 |
| 12. Embryonic Development (15) | 18 |
| 11. Response to Endogenous Stimulus (unranked) | 16 |
| 13. Response to External Stimulus (unranked) | 14 |
| 16. Homeostasis (10) | 14 |
| 14. Sexual Reproduction (14) | 12 |
| 15. Defense Response (unranked) | 12 |
| 16. Pattern Specification (14) | 12 |
| 16. Response to abiotic Stimulus (unranked) | 11 |
| 24. Extracellular Structure organization and Biogenesis (15) | 7 |
| 19. Response to Chemical Stimulus (unranked) | 6 |
| 22. Reproductive Physiological Process (16) | 6 |
| 22. Response to biotic Stimulus (unranked) | 6 |
| 25. Interaction Between Organisms (17) | 6 |
| 20. Growth (13) | 5 |
| 20. Cell Adhesion (11) | 5 |
| 27. Rhythmic Process (14) | 5 |
| 25. Developmental Maturation (16) | 4 |
| 28. Physiological Interaction Between Organisms (18) | 2 |
| 29. Coagulation (unranked) | 2 |
| 30. Segmentation (unranked) | 2 |
| 31. Cell Recognition (13) | 1 |
| 31. Post-embryonic Development (18) | 1 |
| Response to Stimulus (8) | |
| Lysogeny (17) | |
| Pigmentation During Development (18) | |
| Locomotion (12) | |

1. A method of diagnosing bipolar disorder, alcoholism and/or stress disorder in an individual, the method comprising:
determining the level of a plurality of biomarkers for the disorders in a sample from the individual, the plurality of biomarkers selected from the group consisting of biomarkers listed in Table 4 and/or Table 5 and/or 6.

2. The method of claim 1, wherein the plurality of biomarkers comprise a subset of about 17 biomarkers designated as Drd2 (dopamine receptor 2), Clk1 (CDC-like kinase 1), Itgav (integrin alpha V), Gls (glutaminase), Cnp (cyclic nucleotide phosphodiesterase 1), Hnrpdl (heterogeneous nuclear ribonucleoprotein D-like), Kcnj4 (potassium inwardly-rectifying channel, subfamily J, member 4), Gnb1 (guanine nucleotide binding protein, beta 1), Clic4 (chloride intracellular channel 4), Ywhaz (tyrosine 3-monooxygenase/tryptophan 5-monooxygenase activation protein, zeta polypeptide), Sgk (serum/glucocorticoid regulated kinase), Slc38a2 (solute carrier family 38, member 2), Gpm6b (Glycoprotein M6B), Abhd14a (abhydrolase domain containing 14A), Ap1s2 (adaptor-related protein complex 1, sigma 2 subunit), B230337E12Rik (RIKEN cDNA B230337E12 gene), and Snca (synuclein, alpha).

3. The method of claim 1, wherein the plurality of biomarkers comprise a subset of blood biomarkers selected from the group consisting of Cnp (cyclic nucleotide phosphodiesterase 1), Hnrpdl (heterogeneous nuclear ribonucleoprotein D-like), Ywhaz tyrosine 3-monooxygenase/tryptophan 5-monooxygenase activation protein, zeta polypeptide), Sgk (serum/glucocorticoid regulated kinase), Slc38a2 (solute carrier family 38, member 2), Abhd14a (abhydrolase domain containing 14A), Ap1s2 (adaptor-related protein complex 1, sigma 2 subunit), B230337E12Rik (RIKEN cDNA B230337E12 gene), and Snca (synuclein alpha).

4. The method of claim 1, wherein the sample is a bodily fluid.

5. The method of claim 1, wherein the sample is blood.

6. The method of claim 1, wherein the level of the biomarker is determined in a tissue biopsy sample of the individual.

7. The method of claim 1, wherein the level of the biomarker is determined by analyzing the expression level of RNA transcripts.

8. The method of claim 1, wherein the expression level of the biomarker is determined by analyzing the level of protein or peptides or fragments thereof.

9. The method of claim 1, wherein the expression level is determined by an analytical technique selected from the group consisting of microarray gene expression analysis, polymerase chain reaction (PCR), real-time PCR, quantitative PCR, immunohistochemistry, enzyme-linked immunosorbent assays (ELISA), and antibody arrays.

10. The method of claim 1, wherein the determination of the level of the plurality of biomarkers is performed by an analysis of the presence or absence of the biomarkers.

11. A method of predicting the probable course and outcome (prognosis) of bipolar disorder, alcoholism and/or stress disorder in a subject, the method comprising:
analyzing a test sample from a subject, wherein the subject is suspected of having bipolar disorder, alcoholism and/or stress disorder for the presence or level of a plurality of biomarkers, wherein the markers are selected from the group consisting of biomarkers listed in Tables 4-6 and
thereby determining the prognosis of the subject based on the presence or level of the biomarkers and one or more clinicopathological data to implement a particular treatment plan for the subject.

12. The method of claim 11, wherein the clinicopathological data is selected from the group consisting of patient age, previous personal and/or familial history of psychiatric illness, previous personal and/or familial history of response to medications, and any genetic or biochemical predisposition to psychiatric illness.

13. The method of claim 11, wherein the test sample from the subject is of a test sample selected from the group consisting of fresh blood, stored blood, fixed, paraffin-embedded tissue, tissue biopsy, tissue microarray, fine needle aspirates, peritoneal fluid, ductal lavage and pleural fluid or a derivative thereof.

14. (canceled)

15. The method of claim 1 further comprising
selecting a treatment for bipolar disorder, alcoholism and/or stress disorder based on the determination whether the patient suffers from delusion or hallucination.

16. The method of claim 15, wherein the treatment plan is a personalized plan for the patient.

17. A method for clinical screening of agents capable of affecting bipolar disorder, alcoholism and/or stress disorder, the method comprising:
(a) administering a candidate agent to a population of individuals suspected of suffering from bipolar disorder, alcoholism and/or stress disorder;
(b) monitoring the expression profile of one or more of the biomarkers listed in Tables 4-5 in blood samples obtained from the individuals receiving the candidate agent compared to a control group; and
(c) determining that the candidate agent is capable of affecting bipolar disorder, alcoholism and/or stress disorder based on the expression profile of one or more of the biomarkers in the blood samples obtained from the individuals receiving the candidate drug compared to the control.

18. The method of claim 17, wherein the individuals are mice.

19. The method of claim 17, wherein the candidate agent is a pharmaceutical composition.

20. A diagnostic microarray for bipolar disorder, alcoholism and/or stress disorder comprising a plurality of nucleic acid molecules representing genes selected from the group of genes listed in Tables 4-6.

21. (canceled)

22. The diagnostic microarray of claim 20 comprising a panel of biomarkers that are predictive of bipolar disorder, alcoholism and/or stress disorder, wherein the microarray comprises nucleic acid fragments representing biomarkers designated as Cnp (cyclic nucleotide phosphodiesterase 1), Hnrpdl (heterogeneous nuclear ribonucleoprotein D-like), Ywhaz tyrosine 3-monooxygenase/tryptophan 5-monooxygenase activation protein, zeta polypeptide), Sgk (serum/glucocorticoid regulated kinase), Slc38a2 (solute carrier family 38, member 2), Abhd14a (abhydrolase domain containing 14A), Ap1s2 (adaptor-related protein complex 1, sigma 2 subunit), B230337E12Rik (RIKEN cDNA B230337E12 gene), and Snca (synuclein alpha).

23. (canceled)

24. (canceled)

25. (canceled)

26. (canceled)
